(19) United States
(12) Patent Application Publication
(10) Pub. No.: US 2026/0274835 A1
(43) Pub. Date: Sep. 17, 2026

(54) A3 ADENOSINE RECEPTOR POSITIVE ALLOSTERIC MODULATORS

(71) Applicant: The Medical College of Wisconsin, Inc., Milwaukee, WI (US)

(72) Inventors: Kenneth A. Jacobson, Silver Spring, MD (US); Lucas B. Fallot, New Windsor, NY (US); Rama S. Ravi, Rockville, MD (US); Courtney L. Fisher, Milwaukee, WI (US); John A. Auchampach, Milwaukee, WI (US); Veronica Salmaso, Bethesda, MD (US); Balaram Pradhan, Bethesda, MD (US); Robert F. Keyes, Milwaukee, WI (US); Brian C. Smith, Milwaukee, WI (US)

(73) Assignee: The Medical College of Wisconsin, Inc., Milwaukee, WI (US)

(21) Appl. No.: 18/850,144

(22) PCT Filed: Mar. 29, 2023
(86) PCT No.: PCT/US2023/016769

§ 371 Date: Sep. 24, 2024

Related U.S. Application Data
(60) Provisional application No. 63/325,095, filed on Mar. 29, 2022.

(51) Int. Cl. C07D 471/04; A61K 31/4745; A61K 31/496; A61K 31/69; C07B 59/00; C07D 513/04; C07F 5/02

(52) U.S. Cl. CPC C07D 471/04; A61K 31/4745; A61K 31/496; A61K 31/69; C07B 59/002; C07D 513/04; C07F 5/022; C07B 2200/05

(57) ABSTRACT

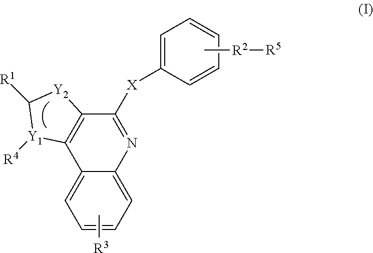

Disclosed are compounds of the formula (I): wherein R¹-R⁵, X, Y₁ and Y₂ are as defined in the specification, pharmaceutical salts thereof and stereoisomers thereof, and pharmaceutical compositions containing one or more of these compounds, salts, or stereoisomers thereof. Also disclosed is a method of treating a having a condition selected from the group consisting of chronic neuropathic pain, heart disease, suppressed immunity, and a disease of the liver, psoriasis, and cancer by administering to the subject having such a condition an effective amount of the compound or pharmaceutical composition.

### CROSS-REFERENCE TO A RELATED APPLICATION

This patent application claims the benefit of U.S. Provisional Patent Application No. 63/325,095, filed Mar. 29, 2022, the disclosure of which is incorporated herein by reference in its entirety for all purposes.

### BACKGROUND OF THE INVENTION

Adenosine receptor (AR) agonists are an important ligand class for drug development due to the many salutary actions of adenosine, such as protection against stress to an organ and tissue repair. Among the beneficial roles of adenosine acting at four subtypes of ARs, activation of the Gi-coupled A₃AR is associated with attenuating chronic neuropathic pain, heart and brain ischemic preconditioning, and anti-inflammatory effects (10-15), without serious cardiovascular side effects. The A₃AR is the only AR subtype overexpressed in immune and cancer cells, adding to its potential as a possible therapeutic target. A₃AR agonists are in Phase 2/3 clinical trials for psoriasis and liver diseases.

Allosteric modulators bind to topographically distinct binding sites from the orthosteric binding site for native agonists and can exert their effects through conformational changes different from orthosteric agonists. Various types of allosteric modulators differ in their pharmacological effects. Positive allosteric modulators (PAMs) amplify the effect of an endogenous agonist at a given GPCR, to enhance signaling with spatiotemporal specificity, which can reduce side effects compared to agonists.

An advantage of PAMs over orthosteric agonists is that they can be event—and site-specific in action. Because adenosine is endogenously elevated in response to localized distress signals within the body, a pure PAM will enhance the protective function of adenosine only when and where it is elevated, i.e., reducing the side effect risk. A second advantage of PAMs over orthosteric ligands is the possibility of achieving a highly selective PAMs for a single AR subtype, assuming that the PAMs bind to a protein region with considerable sequence variability between subtypes. Thirdly, developing PAMs that penetrate the blood-brain barrier (BBB) would be more effective in activating an AR in the central nervous system (CNS). AR agonists have multiple potential pharmaceutical applications in the CNS, but current A₃AR orthosteric agonists, mainly nucleosides, tend to have low BBB permeability, typically 1-2% free passage. Furthermore, it might be possible to induce biased A₃AR allosteric enhancement to select between multiple signaling pathways.

Thus, there is an unmet need for PAMs which are suitable for treating inflammatory and ischemic conditions, cancer, fibrosis, and/or chronic pain.

### BRIEF SUMMARY OF THE INVENTION

The invention provides a compound of formula (I), a pharmaceutically acceptable salt thereof, or a stereoisomer thereof,

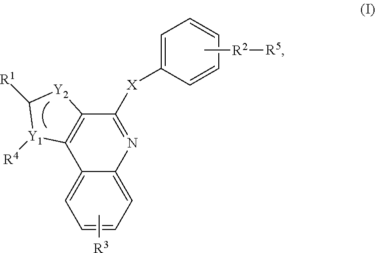

wherein
R¹ is selected from the group consisting of alkyl, cycloalkyl, bicycloalkyl, tricycloalkyl, alkenyl, cycloalkenyl, bicycloalkenyl, tricycloalkenyl, cycloalkoxyalkyl, hydroxycycloalkyl, and cycloalkylcarbonyl, each of which is optionally deuterated, or halogenated, preferably fluorinated;
R² is absent, halo, dihalo, trihalo, SF₅, or a substituent selected from the following groups, each of which is optionally deuterated: alkyl, alkylaminocarbonyl, alkenyl, alkylamino carbonyl alkenyl, hetero bicyclylamino alkyl, carbonyl alkenyl, alkynyl, alkylaminoalkyl alkynyl, heterocyclylaminoalkyl alkynyl, heterocyclyloxy alkynyl, alkylaminocarbonylalkenyl, alkylamino iminoalkenyl, halo thiophenyl alkenyl, oxadizolylalkenyl, imidazolylaryl alkynyl, trialkoxyarylalkenyl, dioxyalrylalkenyl, monoalkoxyarylalkenyl, trifluoromethoxyarylalkenyl, trialkyl tin, and combinations thereof, wherein more than one R² group can be present on the phenyl ring, and wherein each of the R² group, other than halo, dihalo, trihalo, and SF₅, is optionally deuterated and/or halogenated, preferably fluorinated;
R³ is hydrogen, deuterium, and/or halogen, preferably fluorine;
R⁴ is hydrogen or alkyl;
R⁵ is absent, halo, SF₅, or a substituent selected from the group consisting of alkoxycarbonyl, which is optionally deuterated, amino, alkylamino, dialkylamino, trialkylamino, tetraalkylammonio, Boc-amino, guanidino, heterocyclylamino, hetero bicyclylamino, alkylamino imino, halo thiophenyl, oxadizolyl, imidazolylaryl, trialkoxyaryl, dioxyaryl, monoalkoxyaryl, trifluoromethoxyaryl, trialkyl tin, and combinations thereof, each of which is optionally be substituted with a reporter group, linked through an amine, amide, triazole, sulfonamide, urea, or thiourea group;
X is O, S, or NR, wherein R is H or alkyl; and
Y₁ and Y₂ are independently N or NH, O, or S;
wherein the alkyl group is C₁-C₁₂ linear or branched alkyl, the cycloalkyl group is C₃-C₁₀ cycloalkyl group, the cyclic group of the bi- or tri-cycloalkyl is 3-10 membered, the alkenyl group is C₂-C₁₂ linear or branched alkenyl group, the cycloalkenyl group is 3-10 membered cyclic alkenyl group, the bicycloalkenyl and the tricycloalkenyl groups have 4 to 10 membered cycloalkenyl rings, the cycloalkoxyalkyl group can have C₃-C₁₀ cycloalkoxy C₁-C₁₂ linear or branched cycloalkyl group, the hydroxycycloalkyl group includes a hydroxyl group on a C₃-C₁₀ cycloalkyl group, and the cycloalkylcarbonyl includes a C₃-C₁₀ cycloalkyl group.

The invention further provides a pharmaceutical composition comprising a compound or salt of the invention and a pharmaceutically acceptable carrier.

The invention additionally provides a method for activating an A₃ adenosine receptor in a mammal comprising to the mammal an effective amount of a compound or salt of the invention.

The invention also provides a method for treating a subject, wherein the subject has a condition selected from the group consisting of chronic neuropathic pain, heart disease, suppressed immunity, and disease of the liver, psoriasis, and cancer.

The inventive compounds exhibit advantageous absorption, distribution, metabolism, excretion, and toxicity (ADMET) properties.

The binding interactions of PAM for A₃AR occur within the distal portion of TMD7 and helix 8. Non-conserved residue Y284 (7.55) and conserved residue Y293 (8.54) contribute to allosteric PAM activity. In an aspect, compounds with a terminal amino group have interaction with the negatively-charged phosphate groups of the membrane surface. Certain compounds of the invention, for example, 17, enhance the maximal efficacy of A₃ adenosine receptor agonists and increased agonist affinity. Lipidic conjugates of GPCR PAMs improve the pharmacological properties by stabilizing the interaction with hydrophobic anchoring in the bilayer surrounding the receptor protein.

### BRIEF DESCRIPTION OF THE SEVERAL VIEWS OF THE DRAWINGS

FIG. 1 depicts the structures of compounds 8-11 and 12a and 12b, in accordance with an aspect of the invention.

FIG. 2 depicts reaction steps to prepare compounds 13-27, 29, 35, 36, and 39 in accordance with an aspect of the invention.

FIG. 3 depicts additional reaction schemes to prepare compounds shown in FIG. 2.

FIG. 4 depicts reaction schemes to prepare compounds 11-15, 17-20, and 33 in accordance with an aspect of the invention.

FIG. 5 depicts reaction schemes to prepare compounds 35-37 in accordance with an aspect of the invention.

FIG. 6 depicts reaction schemes to prepare compounds 41 and 42 in accordance with an aspect of the invention.

FIG. 7 depicts a reaction scheme to prepare compounds 43 and 44 in accordance with an aspect of the invention.

FIG. 8 depicts reaction schemes to prepare compounds 45-47 in accordance with an aspect of the invention.

FIG. 9 depicts the results of [³⁵S]GTPγS binding assay on compounds 11-13 in accordance with an aspect of the invention.

FIG. 10 depicts the results of [³⁵S]GTPγS binding assay on compounds 21, 17, and 16 in accordance with an aspect of the invention.

FIG. 11 depicts the results of [³⁵S]GTPγS binding assay on compounds 6, 39, and 40 in accordance with an aspect of the invention.

FIG. 12A depicts the results of [³⁵S]GTPYS binding assay on compounds 35 and 41 in accordance with an aspect of the invention.

FIG. 12B depicts the results of [³⁵S]GTPYS binding assay on compounds 42-44 in accordance with an aspect of the invention.

FIG. 12C depicts the results of [³⁵S]GTPγS binding assay on compounds 46 and 47 in accordance with an aspect of the invention.

FIG. 13 depicts a reaction scheme to prepare compounds 52 and 56 in accordance with an aspect of the invention.

FIG. 14 depicts a reaction scheme to prepare compound 98 in accordance with an aspect of the invention.

FIGS. 15A and 15B depict the modulation of EC₅₀ and Eₘₐₓ of agonist Cl-IB-MECA by compound 17 in [³⁵S]GTPYS functional assays and in agonist radioligand binding in accordance with an aspect of the invention.

FIG. 16 depicts the presence of A₃AR structure at the active state, which was visualized by a model generated by homology modeling using an active-state cryo-EM structure of A₁AR (PDB ID: 7LD4) as template.

FIG. 17 depicts the results of [³⁵S]GTPYS binding assay on a compound in accordance with an aspect of the invention.

FIG. 18 depicts the results of [³⁵S]GTPYS binding assay on another compound in accordance with an aspect of the invention.

FIG. 19 depicts the results of [³⁵S]GTPYS binding assay on yet another compound in accordance with an aspect of the invention.

FIG. 20 depicts the results of [³⁵S]GTPγS binding assay on a further compound in accordance with an aspect of the invention.

FIG. 21 depicts the results of [³⁵S]GTPγS binding assay on an yet further compound in accordance with an aspect of the invention.

FIG. 22A and FIG. 22B depict the R² and R³ groups of the compounds in accordance with an aspect of the invention.

FIG. 23 depicts the NMR results obtained on compound 17 in accordance with an aspect of the invention.

FIG. 24 depicts the NMR results obtained on another compound, whose structural formula is shown, in accordance with an aspect of the invention.

FIG. 25 depicts the NMR results obtained on compound 20 in accordance with an aspect of the invention.

### DETAILED DESCRIPTION OF THE INVENTION

In an aspect, the invention provides a compound of the formula (I), a pharmaceutically acceptable salt thereof, or a stereoisomer thereof,

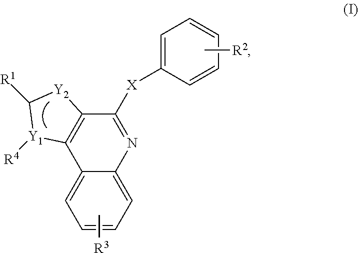

wherein
R¹ is selected from the group consisting of alkyl, cycloalkyl, bicycloalkyl, tricycloalkyl, alkenyl, cycloalkenyl, bicycloalkenyl, tricycloalkenyl, cycloalkoxyalkyl, hydroxycycloalkyl, and cycloalkylcarbonyl, each of which is optionally deuterated, or halogenated, preferably fluorinated;
R² is absent, halo, dihalo, trihalo, SF₅, or a substituent selected from the following groups, each of which is optionally deuterated: alkyl, alkylaminocarbonyl, alkenyl, alkylamino carbonyl alkenyl, hetero bicyclylamino alkyl, carbonyl alkenyl, alkynyl, alkylaminoalkyl alkynyl, heterocyclylaminoalkyl alkynyl, heterocyclyloxy alkynyl, alkylaminocarbonylalkenyl, alkylamino iminoalkenyl, halo thiophenyl alkenyl, oxadizolylalkenyl, imidazolylaryl alkynyl, trialkoxyarylalkenyl, dioxyalrylalkenyl, monoalkoxyarylalkenyl, trifluoromethoxyarylalkenyl, trialkyl tin, and combinations thereof, wherein more than one R² group can be present on the phenyl ring, and wherein each of the R² group, other than halo, dihalo, trihalo, and SF₅, is optionally deuterated and/or halogenated, preferably fluorinated;
R³ is hydrogen, deuterium, and/or halogen, preferably fluorine;
R⁴ is hydrogen or alkyl;
R⁵ is absent, halo, SF₅, or a substituent selected from the group consisting of alkoxycarbonyl, which is optionally deuterated, amino, alkylamino, dialkylamino, trialkylamino, tetraalkylammonio, Boc-amino, guanidino, heterocyclylamino, hetero bicyclylamino, alkylamino imino, halo thiophenyl, oxadizolyl, imidazolylaryl, trialkoxyaryl, dioxyaryl, monoalkoxyaryl, trifluoromethoxyaryl, trialkyl tin, and combinations thereof, each of which is optionally be substituted with a reporter group, linked through an amine, amide, triazole, sulfonamide, urea, or thiourea group;
X is O, S, or NR wherein R is H or alkylenyl; and
Y₁ and Y₂ are independently N or NH, O, or S;
wherein the alkyl group is C₁-C₁₂ linear or branched alkyl, the cycloalkyl group is C₃-C₁₀ cycloalkyl group, the cyclic group of the bi- or tri-cycloalkyl is 3-10 membered, the alkenyl group is C₂-C₁₂ linear or branched alkenyl group, the cycloalkenyl group is 3-10 membered cyclic alkenyl group, the bicycloalkenyl and the tricycloalkenyl groups have 4 to 10 membered cycloalkenyl rings, the cycloalkoxyalkyl group can have C₃-C₁₀ cycloalkoxy C₁-C₁₂ linear or branched cycloalkyl group, the hydroxycycloalkyl group includes a hydroxyl group on a C₃-C₁₀ cycloalkyl group, and the cycloalkylcarbonyl includes a C₃-C₁₀ cycloalkyl group.

In an aspect, the invention provides a compound of formula (I), a pharmaceutically acceptable salt thereof, or a stereoisomer thereof, wherein X is NH, R² is 3,4-dichloro, R³ is H, Y₁ and Y₂ are N, R⁴ is H, and R¹ is selected from the group consisting of

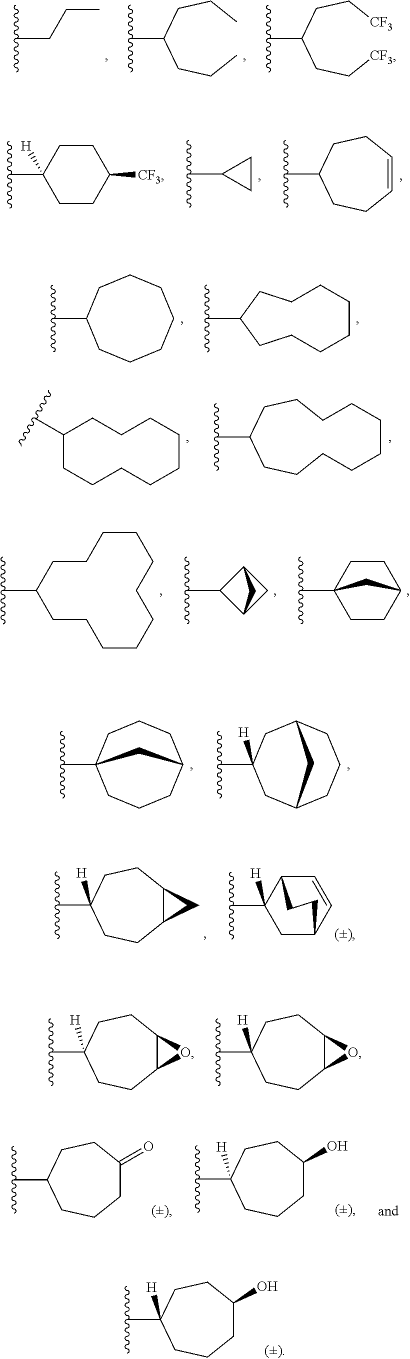

In aspect, the invention provides a compound of formula (I), a pharmaceutically acceptable salt thereof, or a stereoisomer thereof, wherein X is NH, R² is 4-iodo, 4-bromo,

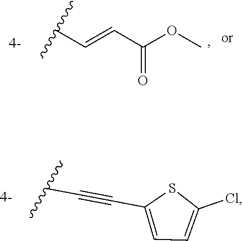

R¹ is cyclohexyl or 4-hept-4-yl, R³ is H, Y₁ and Y₂ are N and NH, and R⁴ is H.

In an aspect, the invention provides a compound of formula (I), a pharmaceutically acceptable salt thereof, or a stereoisomer thereof, wherein the compound is one of the following:

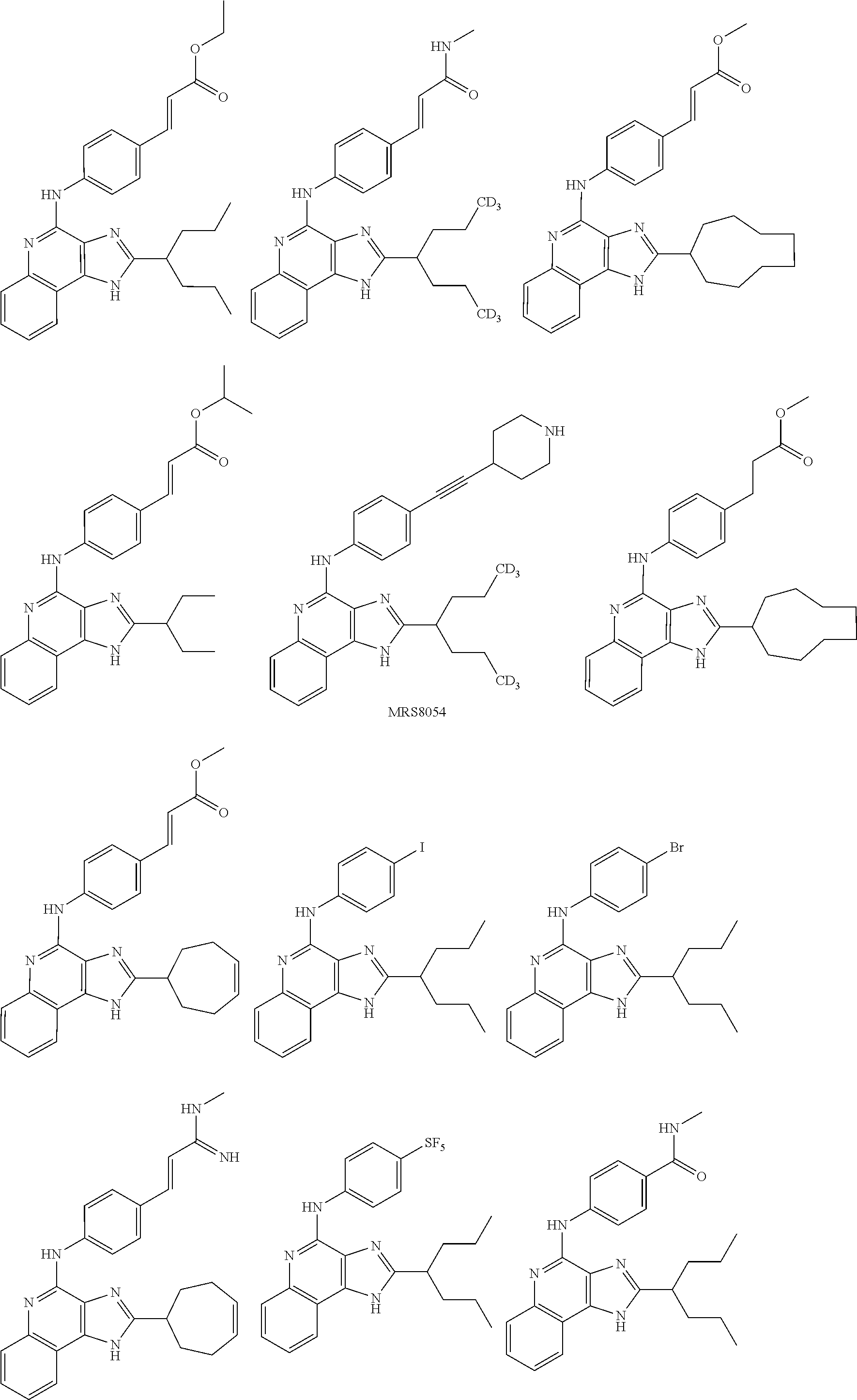

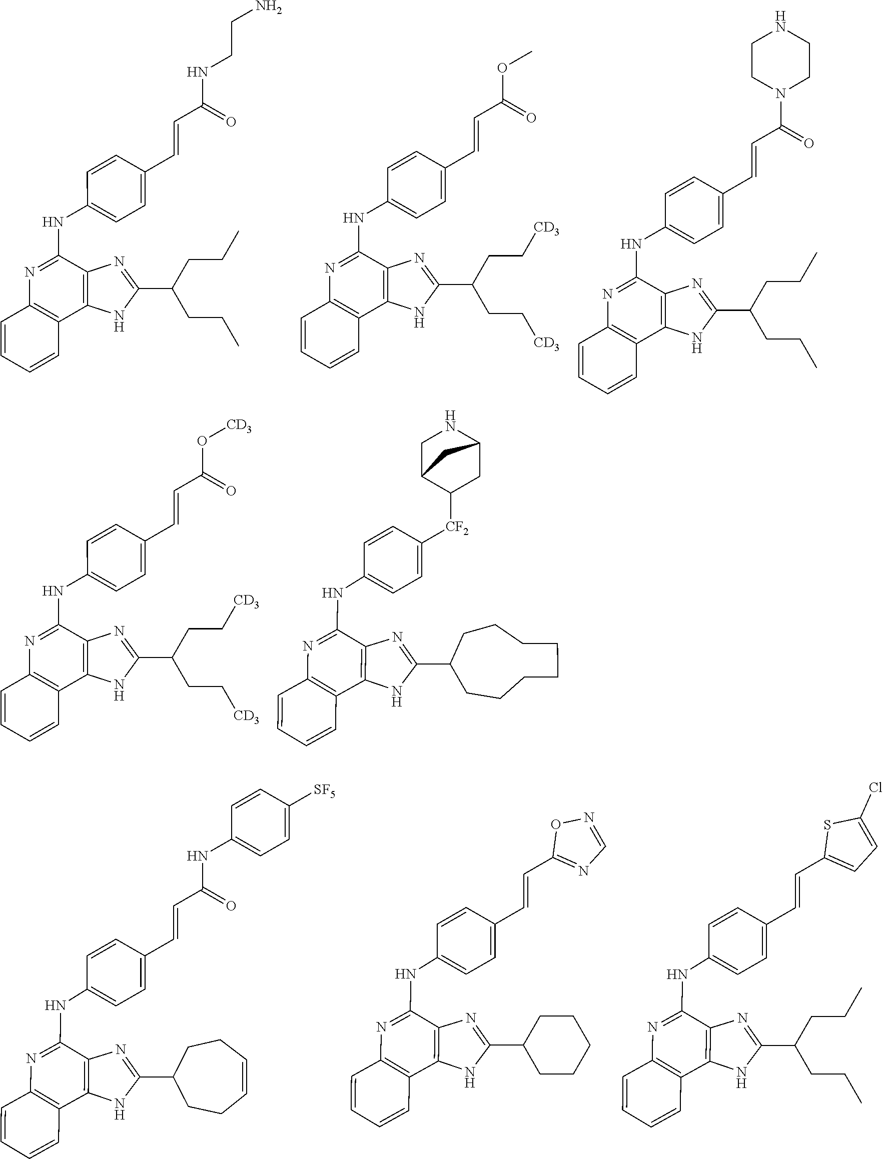

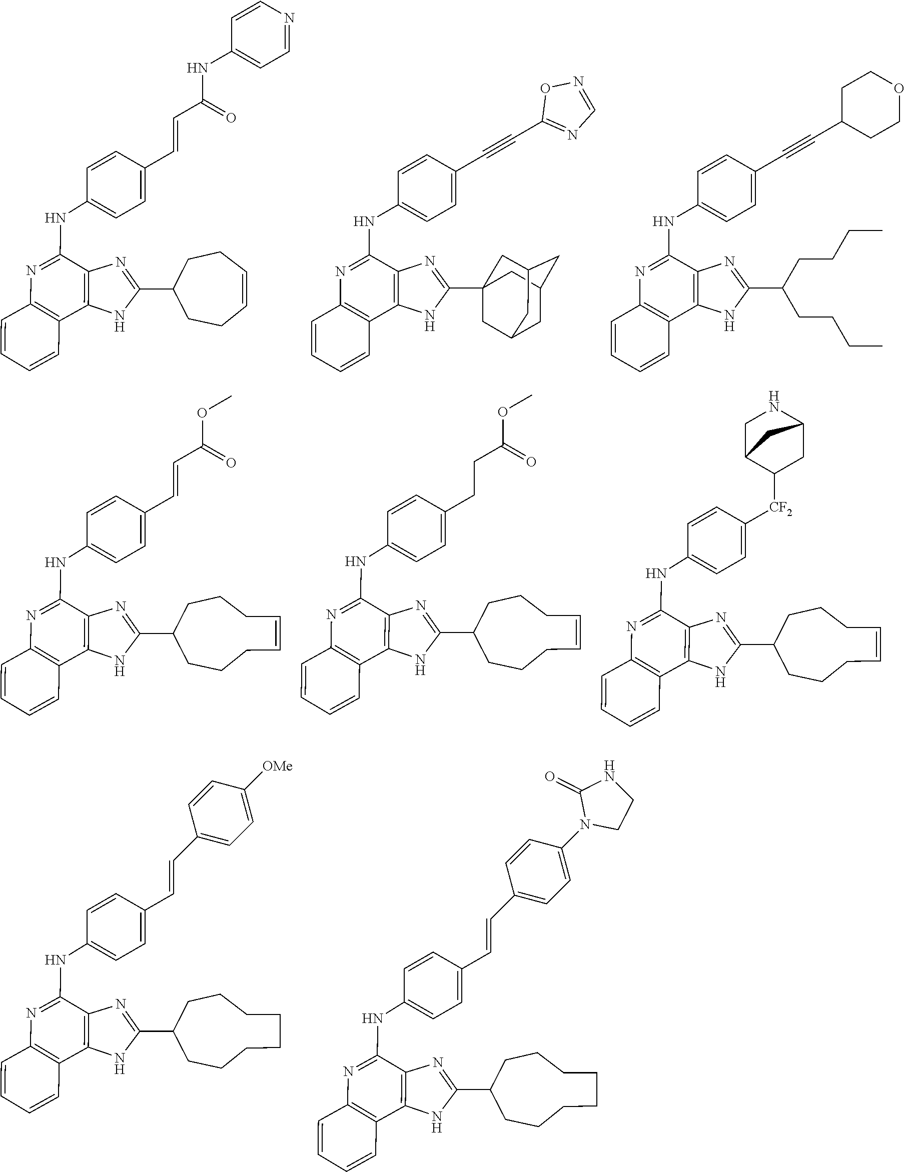

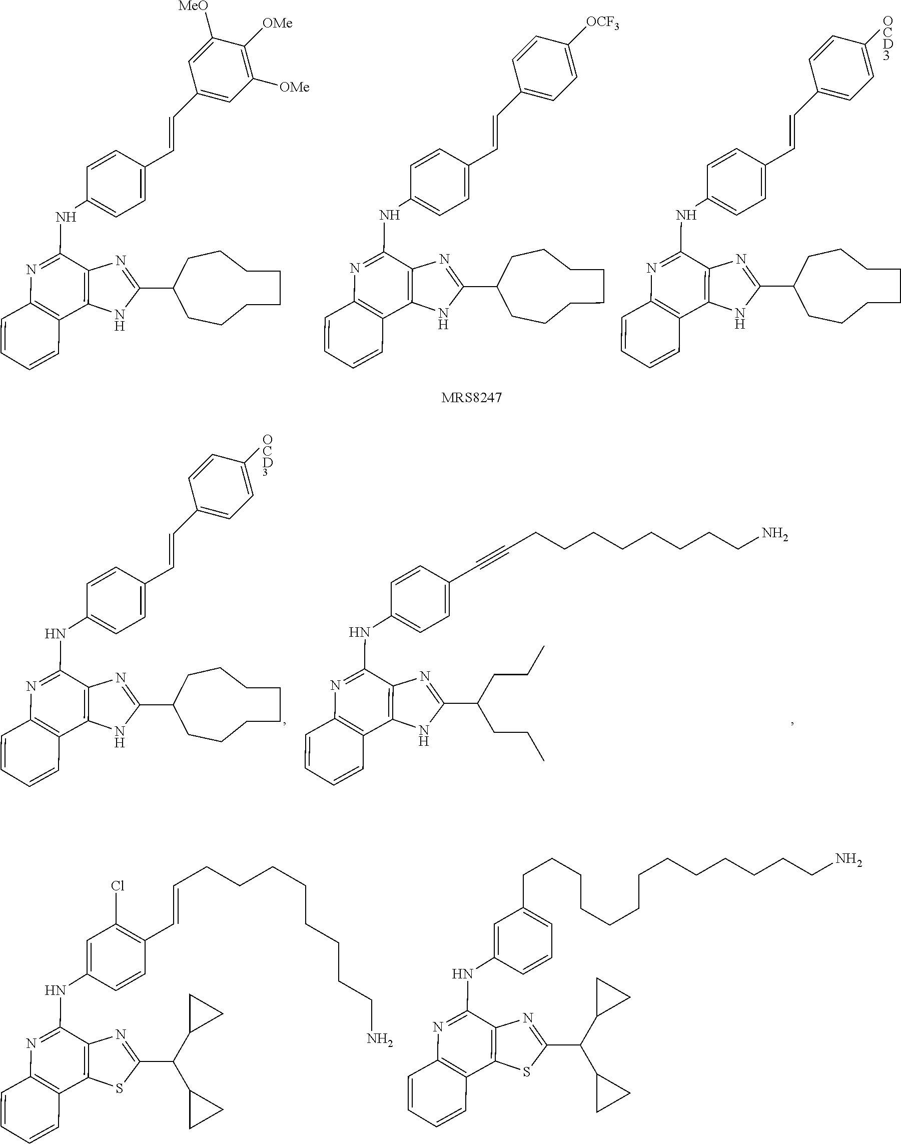

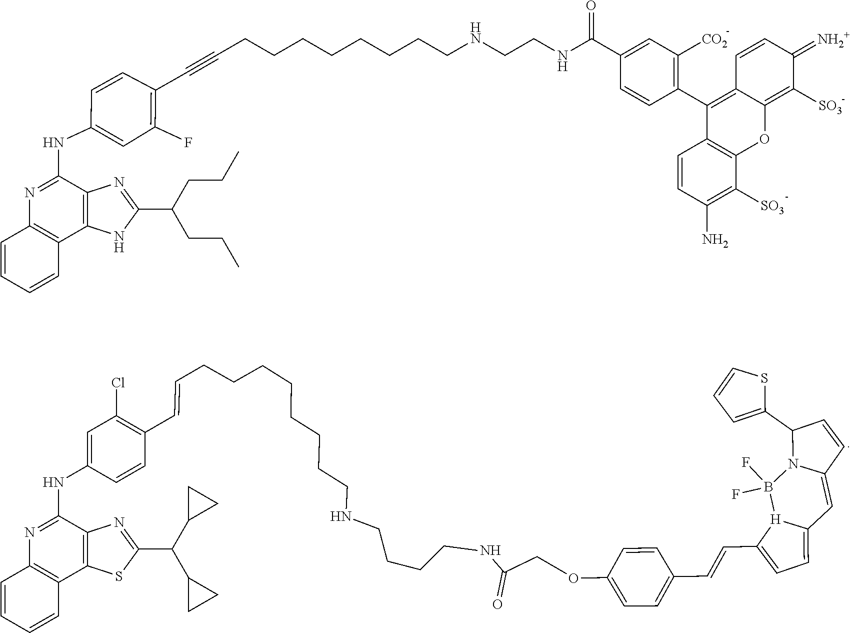

In an aspect, the invention provides a compound of formula (I), a pharmaceutically acceptable salt thereof, or a stereoisomer thereof, wherein the reporter group is a biotin moiety or fluorescent dye.

In an aspect, the invention provides a compound of formula (I), a pharmaceutically acceptable salt thereof, or a stereoisomer thereof, wherein X is NH, Y₁ and Y₂ are N or NH—, R² is aminoalkylamino carbonyl alkenyl, wherein the aminoalkyl moiety is —(CH₂)₂—NH₂ or —(CH₂)₈—NH₂ or wherein R² is alkylamino carbonyl alkenyl, wherein the alkyl moiety is —(CH₂)₅—CH₃, and R³ and R⁴ are H.

In an aspect, the invention provides a compound of formula (I), a pharmaceutically acceptable salt thereof, or a stereoisomer thereof, wherein X is NH, Y₁ and Y₂ are N or NH, and R² is aminoalkyl alkynyl or alkylaminoalkyl alkynyl, which could be attached at o, m, or p-position of the phenyl ring, and R³ and R⁴ are H.

In an aspect, the invention provides a compound of formula (I), a pharmaceutically acceptable salt thereof, or a stereoisomer thereof, wherein R² is p-(CH₂)₈—NH₂, p-(CH₂)₆—NH₂, p-(CH₂)₄—NH₂, p-(CH₂)₈—NHCH₃, or m-(CH₂)₈—NH₂.

In an aspect, the invention provides a compound of formula (I), a pharmaceutically acceptable salt thereof, or a stereoisomer thereof, wherein X is NH, Y₁ and Y₂ are N or NH, and R² aminoalkyl alkenyl or aminoalkyl, and R³ and R⁴ are H.

In an aspect, the invention provides a compound of formula (I), a pharmaceutically acceptable salt thereof, or a stereoisomer thereof, wherein R² is —CH═CH—(CH₂)₈—NH₂ or —(CH₂)₁₀—NH₂.

In an aspect, the invention provides a compound of formula (I), a pharmaceutically acceptable salt thereof, or a stereoisomer thereof, wherein X is NH, Y₁ and Y₂ are N or NH, R¹ is alkyl or cycloalkyl, R² mono or dihalo, and R³ and R⁴ are H.

In an aspect, the invention provides a compound of formula (I), a pharmaceutically acceptable salt thereof, or a stereoisomer thereof, wherein R¹ is cyclohexyl, heptan-4-yl, nonan-5-yl, or di(cyclopropylmethyl)CH—, and R² is 3,4-dichloro or 4-iodo, and R⁵ is absent.

In an aspect, the invention provides a compound of formula (I), a pharmaceutically acceptable salt thereof, or a stereoisomer thereof, wherein X is O, S, or NH, Y₁ and Y₂ are N or NH, R¹ is alkyl or cycloalkyl, R² mono or dihalo, R³ is H, and R⁴ is H, alkyl, arylalkyl, or hydroxyalkyl, and R⁵ is absent.

In an aspect, the invention provides a compound of formula (I), a pharmaceutically acceptable salt thereof, or a stereoisomer thereof, wherein R¹ is heptan-4-yl, cyclohexyl, nonan-5-yl, or di(cyclopropylmethyl)CH—, and R² is H, R³ is H, and R⁴ is methyl, ethanyl-1-ol, benzyl, 3,4-dichloro, 4-iodo, or 3-chloro-4-iodo, and R⁵ is absent.

In an aspect, the invention provides a compound of formula (I), a pharmaceutically acceptable salt thereof, or a stereoisomer thereof, wherein X is NH, Y₁ is N, Y₂ is S or O, R¹ is cyclohexyl, R² is 3,4-dichloro, R⁵ is absent, and R³ and R⁴ are H.

In an aspect, the invention provides a compound of formula (I), a pharmaceutically acceptable salt thereof, or a stereoisomer thereof, wherein X is O, S, or NR, wherein R is methyl, Y₁ and Y₂ are N or NH, R¹ is cyclohexyl, R² is 3,4-dichloro, R⁵ is absent, and R³ and R⁴ are H.

The present invention further provides a pharmaceutical composition comprising a compound, salt, or stereoisomer as described above and a pharmaceutically acceptable carrier. In an aspect, the pharmaceutically acceptable carrier that forms an emulsion, hydrogel, liposome, lipid nanoparticle, and/or micelle in combination with the compound, a pharmaceutically acceptable salt thereof, or a stereoisomer thereof.

The present invention further provides a method of treating a subject for a condition which responds to modulation of A₃ adenosine receptor (A₃AR), the method comprising administering an effective amount of a compound, salt, or stereoisomer as described above.

In an aspect of the above method, the subject has a condition selected from the group consisting of chronic neuropathic pain, heart disease, suppressed immunity, disease of the liver, psoriasis, and cancer.

The present invention also provides a compound as described above, a pharmaceutically acceptable salt, or a stereoisomer thereof, for use in the treatment of a condition which responds to modulation of A₃ adenosine receptor (A₃AR).

The present invention further provides a method of preparing a compound of the invention, the method comprising:
(i) nitrating a dihydroxy compound of the formula

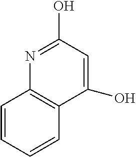 [SMILES: Oc1cc(O)c2ccccc2n1]

by reaction with concentrated nitric acid to obtain a nitrated dihydroxy compound of the formula

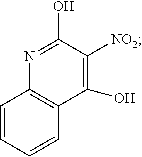 [SMILES: O=[N+]([O-])c1c(O)nc2ccccc2c1O]

(ii) converting the hydroxy groups of the compound from step (i) to chloro groups by reaction with a phosphorous oxychloride at an elevated temperature to obtain a compound of the formula:

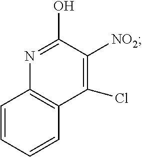 [SMILES: O=[N+]([O-])c1c(O)nc2ccccc2c1Cl]

(iii) reacting the compound from (ii) with a composition comprising aqueous ammonia to obtain the monochloro compound of the formula:

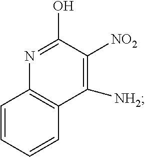 [SMILES: Nc1c([N+](=O)[O-])c(O)nc2ccccc12]

(iv) reducing the nitro group of the compound obtained in (iii) to obtain a diamine of the formula:

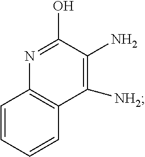 [SMILES: Nc1c(O)nc2ccccc2c1N]

(v) reacting the compound from (iv) with R¹—COOH to obtain a compound of the formula:

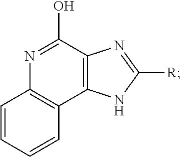

and
(vi) reacting the compound from (v) with an amine to obtain a compound of formula:

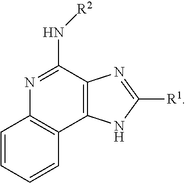

Referring now to terminology used generically herein, the term “alkyl” means a straight-chain or branched alkyl substituent containing from, for example, 1 to about 18 carbon atoms or more, and in particular, from 1 to 4 carbon atoms, from 1 to 6, 1 to 8, 1 to 10, 1 to 12, 1 to 14, or 1 to 16 carbon atoms. Examples of such substituents include methyl, ethyl, propyl, isopropyl, n-butyl, sec-butyl, isobutyl, tert-butyl, pentyl, isoamyl, hexyl, heptyl, octyl, nonyl, decyl, undecyl, dodecyl, and the like.

The term “cycloalkyl,” as used herein, means a cyclic alkyl substituent containing from, for example, about 3 to about 8 carbon atoms, preferably from about 4 to about 7 carbon atoms, and more preferably from about 4 to about 6 carbon atoms. Examples of such substituents include cyclopropyl, cyclobutyl, cyclopentyl, cyclohexyl, cycloheptyl, cyclooctyl, and the like. The cyclic alkyl groups may be unsubstituted or further substituted with alkyl groups such as methyl groups, ethyl groups, and the like.

The term “heterocyclyl,” as used herein, refers to a monocyclic or bicyclic 5- or 6-membered ring system containing one or more heteroatoms selected from the group consisting of O, N, S, and combinations thereof. The heterocyclyl group can be any suitable heterocyclyl group and can be an aliphatic heterocyclyl group, an aromatic heterocyclyl group, or a combination thereof. The heterocyclyl group can be a monocyclic heterocyclyl group or a bicyclic heterocyclyl group. Suitable heterocyclyl groups include morpholine, piperidine, tetrahydrofuryl, oxetanyl, pyrrolidinyl, and the like. Suitable bicyclic heterocyclyl groups include monocylic heterocyclyl rings fused to a C₆-C₁₀ aryl ring. When the heterocyclyl group is a bicyclic heterocyclyl group, both ring systems can be aliphatic or aromatic, or one ring system can be aromatic and the other ring system can be aliphatic as in, for example, dihydrobenzofuran. The term “heteroaryl” refers to a monocyclic or bicyclic 5- or 6-membered ring system as described herein, wherein the heteroaryl group is unsaturated and satisfies Hückel's rule. Non-limiting examples of suitable heteroaryl groups include furanyl, thiopheneyl, pyrrolyl, pyrazolyl, imidazolyl, 1,2,3-triazolyl, 1,2,4-triazolyl, isoxazolyl, oxazolyl, isothiazolyl, thiazolyl, 1,3,4-oxadiazol-2-yl, 1,2,4-oxadiazol-2-yl, 5-methyl-1,3,4-oxadiazole, 3-methyl-1,2,4-oxadiazole, pyridinyl, pyrimidinyl, pyrazinyl, pyridazinyl, triazinyl, benzofuranyl, benzothiopheneyl, indolyl, quinolinyl, isoquinolinyl, benzimidazolyl, benzoxazolinyl, benzothiazolinyl, and quinazolinyl. The heterocyclyl or heteroaryl group is optionally substituted with 1, 2, 3, 4, or 5 substituents as recited herein such as with alkyl groups such as methyl groups, ethyl groups, and the like, halo groups such as chloro, or hydroxyl groups, with aryl groups such as phenyl groups, naphthyl groups and the like, wherein the aryl groups can be further substituted with, for example halo, dihaloalkyl, trihaloalkyl, nitro, hydroxy, alkoxy, aryloxy, amino, substituted amino, alkylcarbonyl, alkoxycarbonyl, arylcarbonyl, aryloxycarbonyl, thio, alkylthio, arylthio, and the like, wherein the optional substituent can be present at any open position on the heterocyclyl or heteroaryl group, or with benzo groups, to form a group of, for example, benzofuran.

The term “alkylcarbonyl,” as used herein, refers to an alkyl group linked to a carbonyl group and further linked to a molecule via the carbonyl group, e.g., alkyl-C(═O)—. The term “alkoxycarbonyl,” as used herein, refers to an alkoxy group linked to a carbonyl group and further linked to a molecule via the carbonyl group, e.g., alkyl-O—C(═O)—.

The term “halo” or “halogen,” as used herein, means a substituent selected from Group VIIA, such as, for example, fluorine, bromine, chlorine, and iodine.

The term “aryl” refers to an unsubstituted or substituted aromatic carbocyclic substituent, as commonly understood in the art, and the term “C₆-C₁₀ aryl” includes phenyl and naphthyl. It is understood that the term aryl applies to cyclic substituents that are planar and comprise 4n+2 π electrons, according to Hückel's Rule.

Whenever a range of the number of atoms in a structure is indicated (e.g., a C₁-C₁₂, C₁-C₈, C₁-C₆, C₁-C₄, or C₂-C₁₂, C₂-C₈, C₂-C₆, C₂-C₄ alkyl, alkenyl, alkynyl, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate). Similarly, the recitation of a range of 6-10 carbon atoms (e.g., C₆-C₁₀) as used with respect to any chemical group (e.g., aryl) referenced herein encompasses and specifically describes 6, 7, 8, 9, and/or 10 carbon atoms, as appropriate, as well as any sub-range thereof (e.g., 6-10 carbon atoms, 6-9 carbon atoms, 6-8 carbon atoms, 6-7 carbon atoms, 7-10 carbon atoms, 7-9 carbon atoms, 7-8 carbon atoms, 8-10 carbon atoms, and/or 8-9 carbon atoms, etc., as appropriate).

The phrase “pharmaceutically acceptable salt” is intended to include nontoxic salts synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, nonaqueous media such as ether, ethyl acetate, ethanol, isopropanol, or acetonitrile are preferred. Lists of suitable salts are found in Remington's Pharmaceutical Sciences, 18th ed., Mack Publishing Company, Easton, PA, 1990, p. 1445, and Journal of Pharmaceutical Science, 66, 2-19 (1977).

Suitable bases include inorganic bases such as alkali and alkaline earth metal bases, e.g., those containing metallic cations such as sodium, potassium, magnesium, calcium and the like. Non-limiting examples of suitable bases include sodium hydroxide, potassium hydroxide, sodium carbonate, and potassium carbonate. Suitable acids include inorganic acids such as hydrochloric acid, hydrobromic acid, hydroiodic acid, sulfuric acid, phosphoric acid, and the like, and organic acids such as p-toluenesulfonic, methanesulfonic acid, benzenesulfonic acid, oxalic acid, p-bromophenylsulfonic acid, carbonic acid, succinic acid, citric acid, benzoic acid, acetic acid, maleic acid, tartaric acid, fatty acids, long chain fatty acids, and the like. Preferred pharmaceutically acceptable salts of inventive compounds having an acidic moiety include sodium and potassium salts. Preferred pharmaceutically acceptable salts of inventive compounds having a basic moiety (e.g., a dimethylaminoalkyl group) include hydrochloride and hydrobromide salts. The compounds of the present invention containing an acidic or basic moiety are useful in the form of the free base or acid or in the form of a pharmaceutically acceptable salt thereof.

It should be recognized that the particular counterion forming a part of any salt of this invention is usually not of a critical nature, so long as the salt as a whole is pharmacologically acceptable and as long as the counterion does not contribute undesired qualities to the salt as a whole.

It is further understood that the above compounds and salts may form solvates, or exist in a substantially uncomplexed form, such as the anhydrous form. As used herein, the term “solvate” refers to a molecular complex wherein the solvent molecule, such as the crystallizing solvent, is incorporated into the crystal lattice. When the solvent incorporated in the solvate is water, the molecular complex is called a hydrate. Pharmaceutically acceptable solvates include hydrates, alcoholates such as methanolates and ethanolates, acetonitrilates and the like. These compounds can also exist in polymorphic forms.

In any of the above embodiments, the compound or salt of formula (I) can have at least one asymmetric carbon atom. When the compound or salt has at least one asymmetric carbon atom, the compound or salt can exist in the racemic form, in the form of its pure optical isomers, or in the form of a mixture wherein one isomer is enriched relative to the other. In particular, in accordance with the present invention, when the inventive compounds have a single asymmetric carbon atom, the inventive compounds may exist as racemates, i.e., as mixtures of equal amounts of optical isomers, i.e., equal amounts of two enantiomers, or in the form of a single enantiomer. As used herein, “single enantiomer” is intended to include a compound that comprises more than 50% of a single enantiomer (i.e., enantiomeric excess up to 100% pure enantiomer).

When the compound or salt has more than one chiral center, the compound or salt can therefore exist as a mixture of diastereomers or in the form of a single diastereomer. As used herein, “single diastereomer” is intended to mean a compound that comprises more than 50% of a single diastereomer (i.e., diastereomeric excess to 100% pure diastereomer).

The present invention further provides a pharmaceutical composition comprising a compound as described above and a pharmaceutically acceptable carrier. The present invention provides a pharmaceutical composition comprising a pharmaceutically acceptable carrier and an effective amount, e.g., a therapeutically effective amount, including a prophylactically effective amount, of one or more of the aforesaid compounds, or salts thereof, of the present invention.

The pharmaceutically acceptable carrier can be any of those conventionally used and is limited only by chemico-physical considerations, such as solubility and lack of reactivity with the compound, and by the route of administration. It will be appreciated by one of skill in the art that, in addition to the following described pharmaceutical compositions; the compounds of the present invention can be formulated as inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Topical formulations, including those that are useful for transdermal drug release, are well-known to those of skill in the art and are suitable in the context of the invention for application to skin. Topically applied compositions are generally in the form of liquids, creams, pastes, lotions and gels. Topical administration includes application to the oral mucosa, which includes the oral cavity, oral epithelium, palate, gingival, and the nasal mucosa. In some embodiments, the composition contains at least one active component and a suitable vehicle or carrier. It may also contain other components, such as an anti-irritant. The carrier can be a liquid, solid or semi-solid. In embodiments, the composition is an aqueous solution. Alternatively, the composition can be a dispersion, emulsion, gel, lotion or cream vehicle for the various components. In one embodiment, the primary vehicle is water or a biocompatible solvent that is substantially neutral or that has been rendered substantially neutral. The liquid vehicle can include other materials, such as buffers, alcohols, glycerin, and mineral oils with various emulsifiers or dispersing agents as known in the art to obtain the desired pH, consistency and viscosity. It is possible that the compositions can be produced as solids, such as powders or granules. The solids can be applied directly or dissolved in water or a biocompatible solvent prior to use to form a solution that is substantially neutral or that has been rendered substantially neutral and that can then be applied to the target site. In embodiments of the invention, the vehicle for topical application to the skin can include water, buffered solutions, various alcohols, glycols such as glycerin, lipid materials such as fatty acids, mineral oils, phosphoglycerides, collagen, gelatin and silicone based materials.

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The compounds, salts, or stereoisomers thereof can be used in any suitable dose. Suitable doses and dosage regimens can be determined by conventional range finding techniques. Generally treatment is initiated with smaller dosages, which are less than the optimum dose. Thereafter, the dosage is increased by small increments until optimum effect under the circumstances is reached. For convenience, the total daily dosage may be divided and administered in portions during the day if desired. In proper doses and with suitable administration of certain compounds, the present invention provides for a wide range of responses. Typically the dosages range from about 0.001 to about 1000 mg/kg body weight of the animal being treated/day. For example, in embodiments, the compounds or salts may be administered from about 100 mg/kg to about 300 mg/kg, from about 120 mg/kg to about 280 mg/kg, from about 140 mg/kg to about 260 mg/kg, from about 150 mg/kg to about 250 mg/kg, from about 160 mg/kg to about 240 mg/kg, of subject body weight per day, one or more times a day, to obtain the desired therapeutic effect.

The following examples further illustrate the invention but, of course, should not be construed as in any way limiting its scope.

### EXAMPLE 1

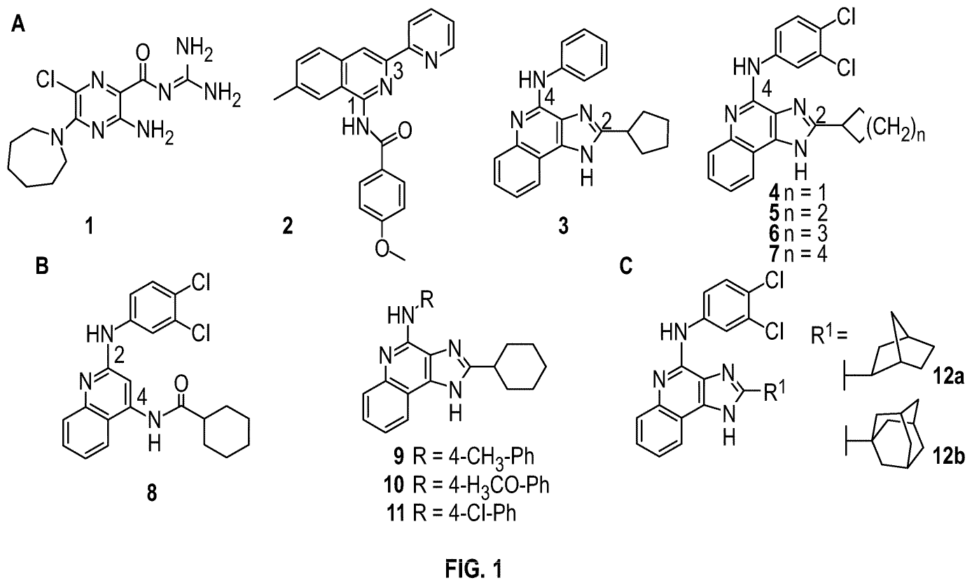

This example illustrates a method of synthesizing compounds of the invention in accordance with an aspect of the invention. Several groups of ¹H-imidazo[4,5-c]quinolin-4-amine derivatives were synthesized for A₃AR pharmacological characterization, for example, as listed in Table 1 (5-8, 13-39), including four known compounds (5-8). The first group of derivatives (5-8, 13-23) had hydrophobic alkyl and cycloalkyl substitutions at the 2 position of the ¹H-imidazo[4,5-c]quinolin-4-amine scaffold with the 3,4-dichlorophenyl group at the 4-amino position. The second group of derivatives (24-29) listed in Table 1 had bridged bicyclic 2 position substitutions with the 3,4-dichlorophenyl group at the 4-amino position, based on the favorable PAM activity of compounds 8 and 19, as well as the exo-norbornanyl and adamantan-1-yl derivatives 12a and 12b (whose formulas are shown in FIG. 1). The third group (30-34) had hydrophilic substitutions introduced on a 2-cycloheptyl ring and a 3,4-dichlorophenyl group at the 4-amino position. The fourth group (35-39) had a cyclohexyl ring or a heptan-4-yl moiety at the 2 position combined with various p-substituted 4-phenylamino groups. Prior studies showed tolerance of 4-methyl, 4-methoxy, and 4-chloro substituted 4-phenylamino substitution (9-11), resulting in promising allosteric enhancement based on a slower dissociation rate and increased Eₘₐₓ of agonists at the A₃AR.18 Compounds 29 and 32-34 were racemic mixtures. 4-(3,4-dichlorophenylamino) substituted compounds were compared to other haloaryl groups (3,5- and 2,4-dichloroaniline derivatives) for the PAM activity, agonist dissociation kinetics, and efficacy. Table 1 lists some of the ¹H-Imidazo[4,5-c]quinolin-4-amine derivatives synthesized and their yields.

TABLE 1
¹H-Imidazo[4,5-c]quinolin-4-amine derivatives synthesized and their
yields

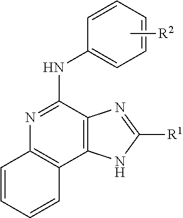

| | | | |
|---|---|---|---|
| | | | Yield^{a} (%, |
| Compound | R² | R¹ | final step) |

2-alkyl and 2-cycloalkyl derivatives

| | | | |
|---|---|---|---|
| 13 | 3,4-Cl₂ | 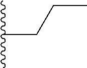 [SMILES: *CCC] | 23^{b} |
| 14 | 3,4-Cl₂ | 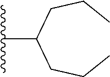 [SMILES: *C(CCC)CCC] | 11^{b} |
| 15 | 3,4-Cl₂ | 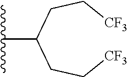 [SMILES: *C(CCC(F)(F)F)CCC(F)(F)F] | 50^{b} |
| 16 | 3,4-Cl₂ | 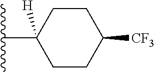 [SMILES: *[C@H]1CC[C@@H](C(F)(F)F)CC1] | 21^{b} |
| 17 | 3,4-Cl₂ | 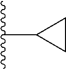 [SMILES: *C1CC1] | 18^{b} |
| 5 | 3,4-Cl₂ | 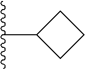 [SMILES: *C1CCC1] | 17^{b} |
| 6 | 3,4-Cl₂ | 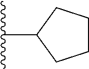 [SMILES: *C1CCCC1] | 23^{b} |
| 7 | 3,4-Cl₂ | 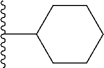 [SMILES: *C1CCCCC1] | 28^{b} |
| 8 | 3,4-Cl₂ | 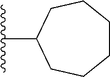 [SMILES: *C1CCCCCC1] | 51^{b} |
| 18 | 3,4-Cl₂ | 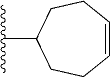 [SMILES: *C1CCC=CCC1] | 23^{b} |
| 19 | 3,4-Cl₂ | 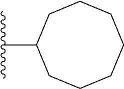 [SMILES: *C1CCCCCCC1] | 27^{b} |
| 20 | 3,4-Cl₂ | 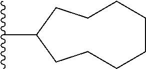 [SMILES: *C1CCCCCCCC1] | 5^{b} |
| 21 | 3,4-Cl₂ | 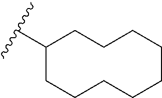 [SMILES: *C1CCCCCCCCC1] | 16^{b} |
| 22 | 3,4-Cl₂ | 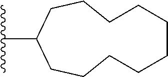 [SMILES: *C1CCCCCCCCCC1] | 15^{b} |
| 23 | 3,4-Cl₂ | 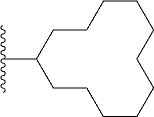 [SMILES: *C1CCCCCCCCCCC1] | 25^{d} |

2-bicycloalkyl derivatives

| | | | |
|---|---|---|---|
| 24 | 3,4-Cl₂ | 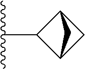 [SMILES: *C1[C@H]2C[C@@H]1C2] | 13^{c} |
| 25 | 3,4-Cl₂ | 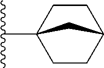 [SMILES: *[C@]12CC[C@H](CC1)C2] | 33^{c} |
| 26 | 3,4-Cl₂ | 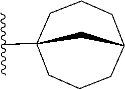 [SMILES: *[C@]12CCC[C@H](CCC1)C2] | 22^{c} |
| 27 | 3,4-Cl₂ | 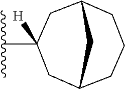 [SMILES: *[C@H]1C[C@H]2CCC[C@@H](C1)C2] | 2^{c} |
| 28 | 3,4-Cl₂ | 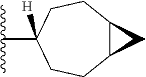 [SMILES: *[C@H]1CC[C@@H]2C[C@@H]2CC1] | 13^{e} |
| 29 | 3,4-Cl₂ | 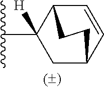 [SMILES: *[C@H]1C[C@H]2C=C[C@@H]1CC2] | 36^{d} |

2-cycloalkyl derivatives with hydrophilic substitution

| | | | |
|---|---|---|---|
| 30 | 3,4-Cl₂ | 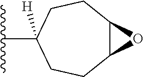 [SMILES: *[C@@H]1CC[C@@H]2O[C@@H]2CC1] | 6^{f} |
| 31 | 3,4-Cl₂ | 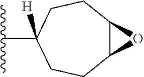 [SMILES: *[C@H]1CC[C@@H]2O[C@@H]2CC1] | 12^{f} |
| 32 | 3,4-Cl₂ | 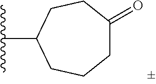 [SMILES: *C1CCCC(=O)CC1] | 23^{g} |
| 33 | 3,4-Cl₂ | 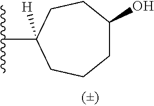 [SMILES: *[C@@H]1CCC[C@H](O)CC1] | 8^{h} |
| 34 | 3,4-Cl₂ | 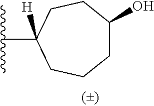 [SMILES: *[C@H]1CCC[C@H](O)CC1] | 12^{h} |

2-alkyl and 2-cycloalkyl derivatives with modified 2-arylamino groups

| | | | |
|---|---|---|---|
| 35 | 4-I | 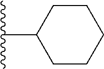 [SMILES: *C1CCCCC1] | 61^{d} |
| 36 | 4-Br | 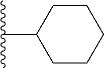 [SMILES: *C1CCCCC1] | 10^{d} |
| 37 | 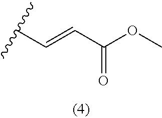 [SMILES: */C=C/C(=O)OC] | 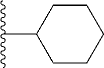 [SMILES: *C1CCCCC1] | 18ⁱ |
| 38 | 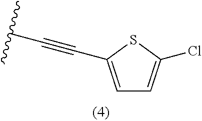 [SMILES: *C#Cc1ccc(Cl)s1] | 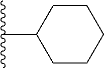 [SMILES: *C1CCCCC1] | 10ⁱ |
| 39 | 4-I | 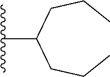 [SMILES: *C(CCC)CCC] | 11^{d} |

a. Isolated yield.
b. Final general procedure C (viii) was used.
c. Final general procedure D (ix) was used.
d. Final general procedure E (x) was used.
e. 18, Et₂Zn, CH₂I₂ (xi).
f. 18, m-CPBA, CHCl₃ (xii).
g. 18, (CH₃)₂S•BH₃, THF, NaOH, H₂O₂, 0° C. (xiii).
h. 33 and 34, DMP, CHCl₃ (xiv).
i. 36, Pd(OAc)₂, CH₂═CHCOOCH₃, Et₃N, 140° C. (xv).
j. 35, Pd(Ph₃P)₂Cl₂, C₆H₃ClS, Cul, Et₃N, 80° C. (xvi).

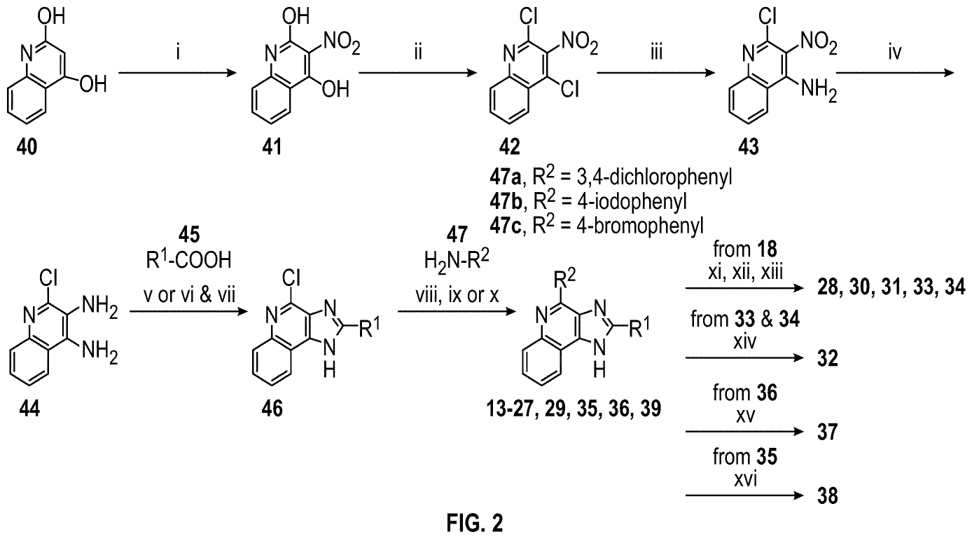

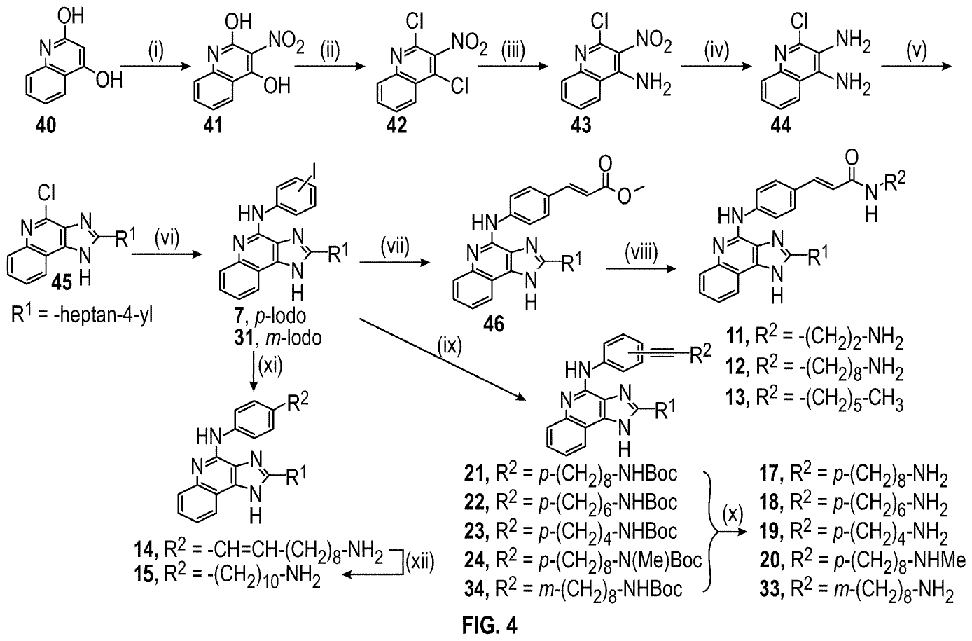

3,4-Diaminoquinoline intermediates 40, 13, 14, and 16 shown in FIGS. 2 and 4 were prepared based on literature procedures. However, a shorter synthesis than the previously reported 9-step route, requiring only 6 steps, was developed to create a new series of ¹H-imidazo[4,5-c]quinolin-4-amine derivatives. The first four steps closely followed a reported synthetic route used to synthesize a related series of 2-(p-substituted-phenyl)-4-phenyl-1H-imidazo[4,5-c]quinoline derivatives, except having 4-phenyl instead of 4-aminophenyl substitution. 23

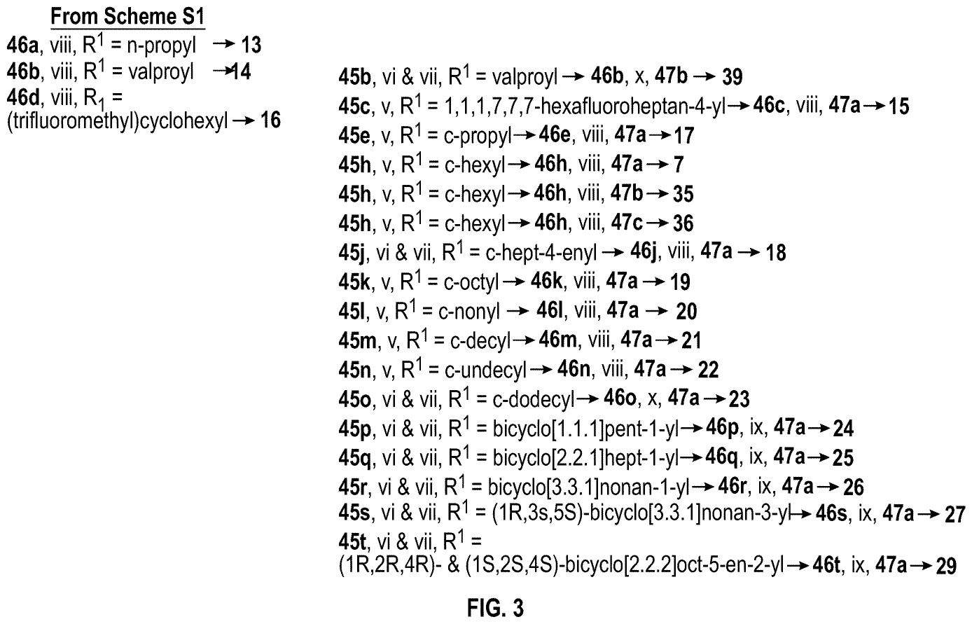

As shown in FIG. 2-3, key intermediates, 7 and 31, were synthesized via a microwave assisted reaction in ethanol at 130° C., in 75-80% yield. Compounds 47 and 14, having a methyl acrylate and amino-decenyl group, respectively, at the 4-position of the phenylamino moiety, were generated from a Heck reaction between compound 7 and methyl acrylate or N-Boc-dec-9-en-1-amine using a Pd (OAc) 2 catalyst as the E-isomer, in a 75-80% yield. Pd-C-induced catalytic transfer hydrogenation with triethylsilane of compound 14 afforded 15 in a quantitative yield. Compounds 21-24 and 34 were prepared by a Sonogashira reaction between compound 7 or 31 and N-Boc-alkyn-1-amine using a bis(triphenylphosphine) palladium II dichloride catalyst. Deprotection of amine group with TFA at room temperature produce compound 17-20 and 33 in 60-70% yield.

Reagents and conditions (FIG. 3): (i) HNO₃, 75° C., 95%; (ii) PhPOCl₂, 135° C., 87%; (iii) 28% aq. NH₃, CH₃CN, 50° C., 97%; (iv) Fe powder, HCl, CH₃CH₂OH/H₂, 75° C., 70%; (v) PPA, R¹—COOH), 120° C., 77%; (vi) iodoaniline, ethanol, MW, 130° C., 75-80%; (vii) methyl acrylate, Pd(OAc)₂, Et₃N, 80° C., 80%; (viii) R²—NH₂, RT−55° C.-130° C., 20-30%; (ix) N-Boc-alkyn-1-amine, PdCl₂ (PPh₃)₂, Cul, TEA, DMF, RT, 75-80%; (x) TFA, DCM, RT, 60-70%; (xi) (a)N-Boc-alken-1-amine, PdC12 (PPh₃) 2, Cul, TEA, DMF, 75%; (b) TFA, DCM, 70%; (xii) Et₃SiH, 10% Pd/C, MeOH, 95%.

Reagents and conditions (FIG. 4): (i) PPA, R¹—COOH), 120° C., 77%; (ii) 3,4-dichloroaniline or 4-iodoaniline in ethanol, or [phenol or 3,4-dichlorophenol or 3,4-dichlorothiophenol in dioxane, Cs₂CO₃], MW, 130° C., 75-80%; (iii) CH₃I, BnBr or 2-bromoethanol, K₂CO₃, acetone, RT−50° C., 25-80%.

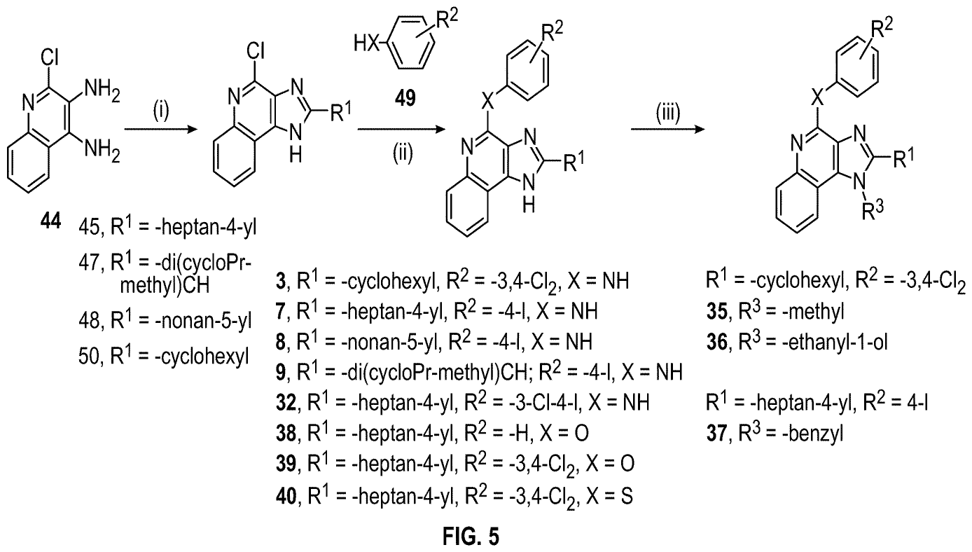

Reagents and conditions (FIG. 5): A. (i) cyclohexanecarbonyl chloride, DMF/DCM, 0° C., 72%; (ii) hexachloroethane, PPh₃, Et₃N, DCM, RT, 69%; (iii) mCPBA, CHCl₃, RT, 81%; (iv) 3,4-dichloroaniline, Tf₂O, ACN, RT, 64%. B. Reagents and conditions: (i) P₂S₅, Py, reflux, 82% (ii) mCPBA, CHCl₃, RT, 92%; (iii) 3,4-dichloroaniline, Tf₂O, ACN, RT, 54%.

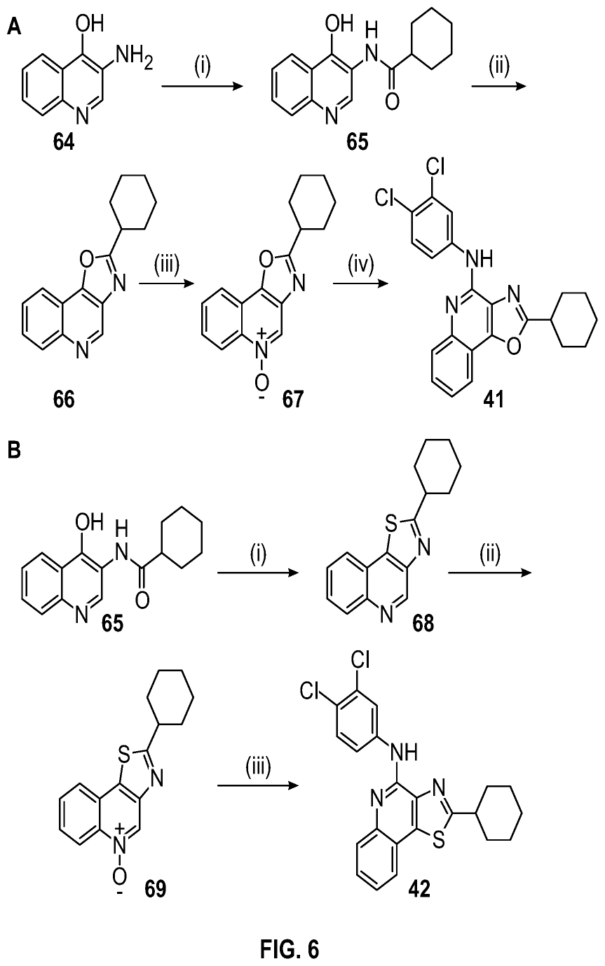

Reagents and conditions (FIG. 6): A. (i) cyclohexanecarbonyl chloride, Py, 0° C., 22%; (ii) NBS, AIBN, anh. benzene, reflux, 90%; (iii) K2CO₃, Cul, Py, MW, 140° C., 51%; (iv) mCPBA, CHCI3, RT, 77%; (v) 3,4-dichloroaniline, Tf₂O, ACN, RT, 48%. B. (i) I2, t-BuOOH, ACN, 80° C., 23%; (ii) cyclohexanecarboxylic anhydride, 160° C., 57%; (iii) (a) Na₂S.9H₂O, CuI, DMF, 80° C., (b) HCl, RT, 54%; (iv) mCPBA, CHCI3, RT, 97%; (v) 3,4-dichloroaniline, Tf₂O, ACN, RT, 87%.

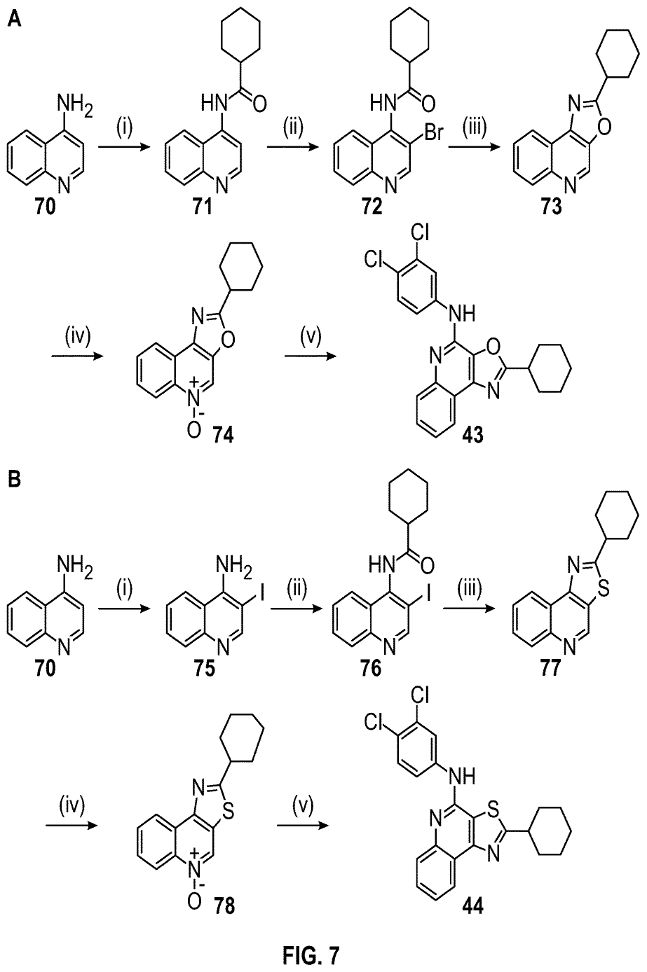

Reagents and conditions (FIG. 7): (i) NH₄OH, dioxane, 140° C., 100%; (ii) 10% Pd/C, H₂, THF/MeOH, RT; (iii) cyclohexanecarboxylic acid, PPA, 180° C., 74%; (iv) mCPBA, DCM/CHCl₃: MeOH, reflux, 86%; (v) 3,4-dichloro-N-methylaniline, Tf₂O, ACN, RT, 68%; (vi) 3,4-dichlorophenol, PyBroP, Ag₂CO₃, DCE, 70° C., 78%; (vii) 3,4-dichlorobenzenethiol, Tf₂O, ACN, RT, 53%.

In the first step of the six-step route shown in FIG. 3, quinoline-2,4-diol 40 was treated with nitric acid to produce 3-nitroquinoline-2,4-diol 41. 41 was then chlorinated with phenylphosphonic dichloride to afford 2,4-dichloro-3-nitroquinoline 42. The step-2 product was then aminated with 28% aqueous ammonia to give 2-chloro-3-nitroquinolin-4-amine 43. Subsequently, Fe powder and hydrochloric acid reduced the 3-nitro group to an amine to provide the vicinal diamine, 2-chloroquinoline-3,4-diamine 44.

Two alternative step-5 protocols cyclized the vicinal diamine with a carboxylic acid. See FIG. 4. The first reaction protocol, general procedure A (v), utilized polyphosphoric acid (PPA) for the condensation between 2-chloroquinoline-3,4-diamine and the appropriate carboxylic acid followed by the cyclization to the imidazole 46. The second reaction protocol, general procedure B (vi), required two steps. The reaction first made an adduct between the coupling agent tetramethylchloroformamidinium hexafluorophosphate (TCFH) and N-methylimidazole (NMI), forming an acyl imidazolium electrophile with the appropriate carboxylic acid The vicinal diamine 44 subsequently reacted with the electrophile to produce an amide intermediate (not shown). The published method uses room temperature. However, it was found that heating at 60° C. brought the reaction to completion, increasing the yield. The crude amide was subjected to a base-catalyzed cyclization reaction (vii) to form the imidazole ring in 46 and incorporate a 2 position substitution on the quinoline scaffold.

The last step, a C—N cross-coupling reaction, was performed using three different reaction protocols. The first protocol utilized the palladium catalyst tris(dibenzylideneacetone) dipalladium (0) (Pd₂ (dba)₃) (general procedure C-(viii)), while the second used a water-activated palladium acetate (Pd(OAc)₂) catalyst (general procedure D-(ix)). The third reaction protocol was a microwave-assisted reaction in ethanol at 130° C. to achieve the final ¹H-imidazo[4,5-c]quinolin-4-amine derivative (general procedure E-(x)).

### EXAMPLE 2

This example illustrates synthesis compounds in accordance with an aspect of the invention.

Compounds 8-9, 32 and 38-40 were synthesized, as shown in FIG. 3, via microwave assisted reaction in ethanol or dioxane with Cs₂CO₃ (in case of phenol or thiophenol) at 130° C., in 75-80% yield. N-alkylation of the imidazole moiety in acetone or DMF in presence of K₂CO₃ produced compounds 35-37 in 25-80% yield.

FIG. 5 depicts the synthesis of 1-oxo- (41) and 1-thio-substituted (42) 4-(halophenylamino) analogues of 2,4-disubstituted ¹H-imidazo[4,5-c]quinoline, as shown in FIG. 5.

FIG. 6 depicts the synthesis of 3-oxo- (43) and 3-thio-substituted (44) 4-(halophenylamino) analogues of 2,4-disubstituted ¹H-imidazo[4,5-c]quinolines.

FIG. 7 depicts the synthesis of 4-methylamino (45), 4-oxo- (46), and 4-thio-substituted (47) 4-(3,4-dichlorophenylamino) analogues of 2,4-disubstituted ¹H-imidazo[4,5-c] quinolines.

### EXAMPLE 3

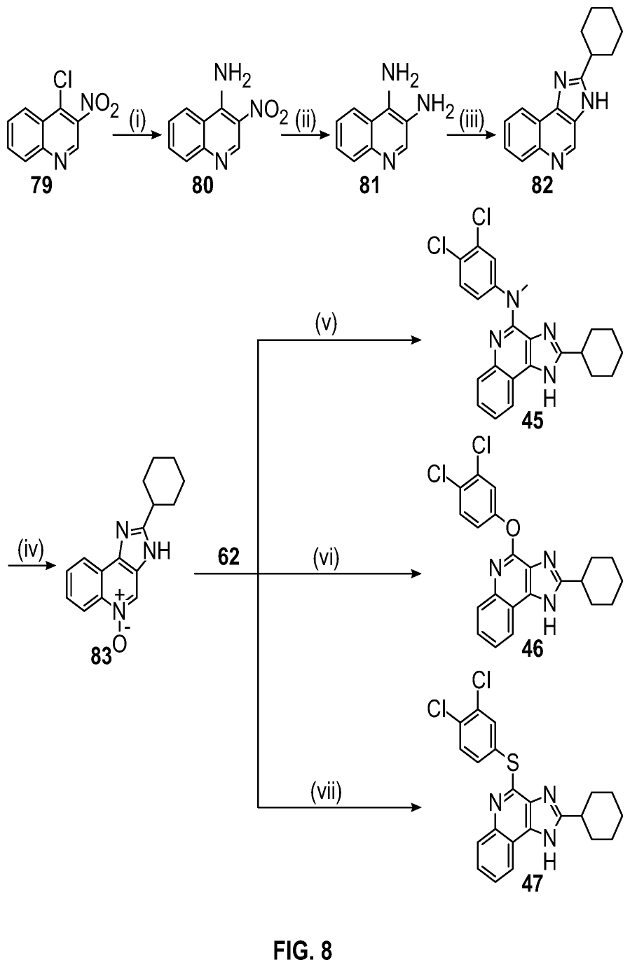

This example illustrates the pharmacological properties of certain compounds of the invention, as set forth in Tables 2-3 and in FIG. 8-14.

Creation of stable HEK293 cell lines expressing wild-type and human/mouse chimeric A₃ARs: The full-length mouse (as previously described²⁵), human (purchased from the cDNA Resource Center), and human/mouse chimeric (custom synthesized by TOP Gene Technologies, St. Laurent, Quebec) A₃AR cDNAs were subcloned into pcDNA3.1. The constructs were transfected into HEK293 cells (American Type Culture Collection, Manassas, VA) using Lipofectamine 2000 reagent (Invitrogen, Waltham, MA) and selected with 2 mg/ml of G418 in cell culture media (DMEM with 10% fetal bovine serum, 100 units/ml penicillin, and 100 μg/ml streptomycin). Cell lines derived from individual clones were maintained in cell culture media containing 0.6 mg/ml G418. The level of receptor expression in each of the cell lines was equivalent (~1,500 fmol/mg) based on saturation radioligand binding analyses (not shown).

Membrane preparations: Transfected HEK293 cells were washed with PBS followed by homogenization in Buffer A containing 10 mM Na+-HEPES, 10 mM EDTA, 1 mM benzamidine (pH 7.4) and centrifugation at 27,000×g for 30 min at 4° C. Cell pellets were subsequently re-homogenized in HE buffer containing 10 mM Na+-HEPES, 1 mM EDTA, 1 mM benzamidine (pH 7.4) and re-centrifuged. The supernatant was discarded, and cell pellets were resuspended in HE buffer containing 10% sucrose and stored at −20° C.

[³⁵S]GTPγS binding assays: Cell membranes (5 μg protein) isolated from transfected HEK293 cells were pre-treated with modulators for one hour in 100 μl GTPγS binding buffer (50 mM Tris HCL [pH 7.4], 1 mM EGTA, 10 mM MgCl₂, 100 mM NaCl, 0.004% CHAPS, and 0.5% BSA) in a 96-well large-volume polypropylene assay plate. In all assays, ZM-241385 (300 nM) and PSB-603 (300 nM) were included to block A₂BARs expressed endogenously in HEK293 cells. Adenosine deaminase (ADA) (1 μl/ml) was also included to degrade any endogenous adenosine that might be produced during the assay, except when adenosine was used as the orthosteric agonist. The reactions were initiated by the addition of ~0.2 nM [³⁵S]GTPγS and agonist in 100 μl GTPγS binding buffer and allowed to incubate for two hours at room temperature. At the end of the two-hour incubation period, the membranes were harvested by rapid filtration through Whatman GF/B filters that had been pre-soaked for two hours in GTPγS binding buffer containing 0.02% CHAPS using a 96-well cell harvester (Brandel, Gaithersburg, MD). Radioactivity trapped in the filters was measured by liquid scintillation counting. Non-specific binding of [³⁵S]GTPγS was determined in the presence of 10 μM unlabeled GTPγS.

Binding assays with [¹²⁵I]I-AB-MECA: Cell membranes (50 μg) isolated from transfected HEK293 cells as described above were incubated in 100 μl binding buffer (50 mM Tris-HCl [pH 7.4], 10 mM MgCl2, 1 mM EDTA, and 1 unit/ml adenosine deaminase) containing ~0.3 nM [¹²⁵I]I-AB-MECA and indicated concentrations of the A₃AR allosteric modulator compounds. The reactions were incubated at room temperature for the times indicated after which bound and free radioligand were separated by rapid filtration through GF/C glass fiber filters. Radioactivity trapped in the filters was measured using a gamma counter. For dissociation studies, [¹²⁵I]IAB-MECA (~0.3 nM) was incubated with HEK 293 cell membranes (50 μg) expressing ARs for 3 h at room temperature in binding buffer (100 μl), after which the assay was begun by the addition of 100 μM adenosine-5′-N-ethylcarboxamide (NECA) along with the enhancer compounds or equivalent vehicle. Specific [¹²⁵I]I-AB-MECA binding was measured by rapid filtration at the indicated time intervals. For equilibration binding assays, membranes (50 μg) were incubated with [¹²⁵I]I-AB-MECA (~0.3 nM) and the modulator compounds for the indicated times before filtration. For all assays, non-specific binding was determined by incubation in the presence of 100 μM NECA. [¹²⁵I]I-AB-MECA was prepared by radioiodination of AB-MECA using the chloramine-T method and purified by HPLC.

Bioluminescence resonance energy transfer (BRET) assays: To measure A₃AR-mediated β-arrestin-2 recruitment by BRET1, HEK293T cells were co-transfected (1:15) with two constructs (pcDNA3.1) encoding the human A₃AR with C-terminally fused Renilla Luciferase (Rluc8) and N-terminally fused Venus-tagged β-arrestin-2 in DMEM supplemented with 10% fetal bovine serum, 100 units/ml penicillin, and 100 μg/ml streptomycin using TransIT-293 transfection reagent (Mirus Bio, Madison, WI). To measure A₃AR G protein coupling via βγ subunit dissociation, as assessed by BRET2 (TRUPATH open-source biosensor plateform28), HEK293T cells were co-transfected in DMEM supplemented with 10% fetal bovine serum, 100 units/ml penicillin, and 100 μg/ml streptomycin with constructs encoding the indicated Rluc8-fused human Gα isoprotein, human Gβ1, C-terminally fused GFP2 fused to human Gγ2, and either the human or mouse A₃AR using TransIT-293. After 24 hours, transfected cells were seeded in poly-l-lysine coated 96-well white-walled clear-bottom cell culture plates at a density of 30,000 cells per well in DMEM with 1% dialyzed fetal bovine serum, 100 units/ml penicillin, and 100 μg/ml streptomycin, and 25 mM HEPES (pH 7.4). The next day, the cell culture medium was aspirated, and cells were washed once with assay buffer (Hank's buffered salt solution with Mg²⁺, Ca²⁺ and 20 mM HEPES [pH 7.4]) and allowed to incubate for one hour at 37° C. in 60 μl fresh assay buffer. Cells were subsequently treated with 30 μl of drugs (3×; agonist and modulator compounds) prepared in drug buffer (Hank's buffered salt solution with Mg²⁺, Ca²⁺, 0.3% BSA, 0.03% ascorbic acid, and 20 mM HEPES [pH 7.4]) as indicated, and incubated for 45 minutes at 37° C. For β-arrestin-2 recruitment assays, 10 μl of the Rluc8 substrate coelenterazine h (Promega, Madison, WI; 5 μM final concentration) was added per well, and the plates were incubated for an additional 15 minutes to allow for cell penetration and immediately read for bioluminescence at 480 nm and fluorescence eYFP emission at 515 nm every five minutes for 30 minutes with a Mithras 940 Multimode Plate Reader. BRET1 ratios were calculated (emission at 515 nm/emission at 480 nm) first, then the NET BRET1 was determined by subtracting the BRET ratio from control wells containing cells transfected with only the donor plasmid (human A₃AR-Rluc8) from the BRET ratio of experimental wells. For BRET² G protein-coupling assays, 10 mL of the RLuc8 substrate coelenterazine 400a (Nanolight Technology, Pinetop, AZ; 5 mM final concentration) was added per well, the plates were incubated for 15 min, and then read for bioluminescence at 385 nM and fluorescent GFP2 emission at 510 nm. NET BRET² ratios were calculated in the same manner as described above for the BRET¹ assays.

Data Analysis: E_{Max} and EC₅₀ values were calculated from data obtained from agonist concentration-response curves according to: E=(E_{Max}>x)/(EC₅₀+x), in which x is the agonist concentration. All values were statistically compared using a two-tailed unpaired Student's t-test or two-tailed paired Student's t-test, as indicated. For dissociation binding assays, fit to a one-phase exponential decay model: Y=(Y₀−NS)^{(-kxt)}, in which Y₀ is specific binding at time 0, k is the dissociation rate constant, and t is the elapsed time. All values are presented as the mean±SEM. A p value <0.05 was considered statistically significant.

To evaluate pure allosteric actions, the effect of the modulators was assessed in a [¹²⁵I]I-AB-MECA dissociation binding assay using transfected HEK293 cell membranes. Cell membranes were incubated with [¹²⁵I]I-AB-MECA for 3 hours, and then binding was determined after the addition of saturating NECA (100 μM), which prevents [¹²⁵I]I-AB-MECA from re-binding after it has dissociated. In the absence of modulator (vehicle), ~25% of the radioligand remained bound 1 hour after the addition of NECA. An increase in binding by a modulator compound indicates an allosteric effect to slow [¹²⁵I]I-AB-MECA dissociation. Using this assay, it was observed that several of the derivatives from both series at a concentration of 10 μM slowed [¹²⁵I]I-AB-MECA dissociation with a SAR similar to that observed in equilibrium binding assays, whereby increasing bulky cycloalkyl substitutions from C-4 to C-8/9 (2-7 and 15-19) produced maximal effects. The bicyclic (except for the adamantyl-substituted quinoline derivative, 22) and especially the heptan-4-yl derivatives (13 and 23) also slowed [¹²⁵I]I-AB-MECA dissociation. Maximal slowing was produced by the 2-heptan-4-yl imidazoquinolin-4-amine modulator (13).

Several interesting observations arise when the dissociation binding data in FIG. 1A and the equilibrium binding data in FIG. 1B are considered together. Because neither compound with 2-cyclopropyl substitution (1, 14) affected the [¹²⁵I]I-AB-MECA dissociation binding rate and therefore lacked allosteric actions, the reduction in equilibrium binding produced in both compound series presumably is caused by competitive antagonism. For other compounds that slowed [¹²⁵I]I-AB-MECA dissociation without increasing equilibrium binding, which occurred with several of the quinoline derivatives (16, 19, 21, and 23), prominent competitive antagonism is assumed.

All compounds that increased equilibrium binding, including compounds 4, 5, 6, 12, and 13 within the imidazoquinolin-4-amine series containing C-6 to C-8 cycloalkyl, bicyclic, or the heptan-4-yl substitutions, produced prominent effects to slow [¹²⁵I]I-AB-MECA dissociation, implicating prominent allosterism with diminished competitive antagonism.

The two series of modulators were evaluated in a [³⁵S]GTPγS binding assay using isolated HEK293 cell membranes expressing the h A₃AR, which detects receptor-induced G protein activation. For these studies, concentration-response curves with the orthosteric agonist Cl-IB-MECA were assessed in the presence of vehicle or 0.1, 1, or 10 μM modulator compound. A clear SAR emerged with each series of compounds that correlated well with the single-point radioligand binding assays. The cyclopropyl derivatives from both chemical series (1, 14) right-shifted the Cl-IB-MECA concentration-response curve in a concentration-dependent manner without altering efficacy, indicative of competitive antagonism. As the ring size increased, efficacy enhancement became apparent, beginning with the C-4 derivatives (2, 15), while potency reduction diminished, correlating with the effect of the modulators in radioligand binding assays to slow [¹²⁵I]I-AB-MECA dissociation while tending to improve [¹²⁵I]I-AB-MECA equilibrium binding. The greatest increase in efficacy enhancement within the imidazoquinolin-4-amine series occurred with the C-6 (4, LUF6000), C-9 (7), and norbornyl (12) derivatives; among these, the C-9 (7) derivative tended to increase the potency of Cl-IB-MECA, whereas the other two reduced it. While the magnitude of the efficacy enhancing action of the heptan-4-ylderivative (13) was not as prominent, it is notable that it appeared to be the most potent among this series given that it produced a maximal degree of efficacy enhancement at a concentration of only 100 nM. In comparison, the lowest concentration to produce maximal activity for the other compounds exhibiting substantial PAM activity including LUF6000 (4) was 1 μM. It is necessary to mention that all efforts to fit the [³⁵S]GTPγS binding data to operation models to calculate affinity constants and cooperativity factors for the modulator compounds were not successful, presumably because data from a greater range and number of concentrations is required for solvation and because standard models do not account for mechanisms leading to reduced agonist potency such as competitive antagonism. Within the quinoline series, our lead compound LUF6096 containing the cyclohexyl substitution (17) produced the greatest increase in efficacy

enhancement without reducing the potency of CI-IB-MECA. Correlating with the [¹²⁵I]I-AB-MECA equilibrium binding data, this series of derivatives in general produced a greater reduction in agonist potency.

The compounds increased the Eₘₐₓ and or functional potency of agonist CI-IB-MECA in a [³⁵S]GTPγS binding assay, performed as in Fisher et al. and Fallot et al. The in vitro activity of the compounds was compared to reference compounds 3-7.

TABLE 2
¹H-Imidazo[4,5-c]quinolin-4-amine and Related Heterocyclic Derivatives
Synthesized and their Allosteric Effect on Agonist-Induced A₃AR Activation. X = NH, unless
noted otherwise.

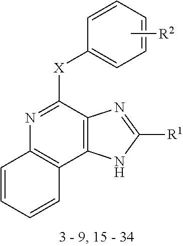

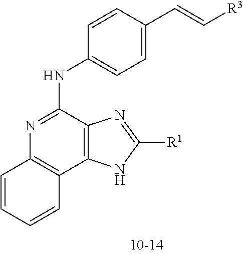

| | | | | |
|---|---|---|---|---|
| Compound | R² or R³ | R¹ | Eₘₐₓ^{a} (%) | IC₅₀ ratio^{b} |

2-alkyl and 2-cycloalkyl derivatives

| | | | | |
|---|---|---|---|---|
| 3 (LUF6000) | 3,4-Cl₂ | 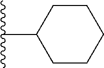 [SMILES: *C1CCCCC1] | 207.3 ± 9.4 | 0.517 |
| 4 (MRS7788) | 3,4-Cl₂ | 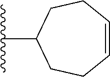 [SMILES: *C1CCC=CCC1] | 227.4 ± 10.9 | 1.49 |
| 5 (MRS7827) | 3,4-Cl₂ | 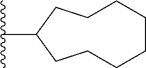 [SMILES: *C1CCCCCCCC1] | 227.5 ± 14.4 | 1.57 |
| 6 (MRS7551) | 3,4-Cl₂ | 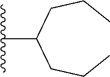 [SMILES: *C(CCC)CCC] | 220.6 ± 14.6 | 2.07 |

NH-phenyl p-substituted derivatives

| | | | | |
|---|---|---|---|---|
| 7 (MRS8054) | 4-I | 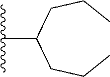 [SMILES: *C(CCC)CCC] | 221.1 ± 11.2 | 1.56 |
| 8 (MRS8260) | 4-I | 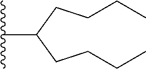 [SMILES: *C(CCCC)CCCC] | | |
| 9 (MRS8285) | 4-I | 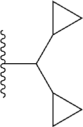 [SMILES: *C(C1CC1)C1CC1] | | |
| 10 (MRS7975) | CO₂CH₃ | 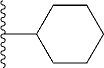 [SMILES: *C1CCCCC1] | 144.9 ± 11.3^{c} | 1.48^{c} |
| 11 (MRS8160) | CONH(CH₂)₂NH₂ | 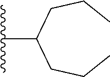 [SMILES: *C(CCC)CCC] | 120.4 ± 7.4 | 0.784 |
| 12 (MRS8205) | CONH(CH₂)₈NH₂ | 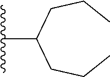 [SMILES: *C(CCC)CCC] | 148.9 ± 6.8 | 0.968 |
| 13 (MRS8204) | CONH(CH₂)₅CH₃ | 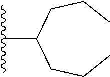 [SMILES: *C(CCC)CCC] | 135.4 ± 10.2 | 0.303 |
| 14 (MRS8263) | (CH₂)₈NH₂ | 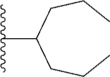 [SMILES: *C(CCC)CCC] | | |
| 15 (MRS8265) | (CH₂)₁₀NH_{2 (4-)} | 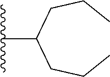 [SMILES: *C(CCC)CCC] | | |
| 16 (MRS8055) | 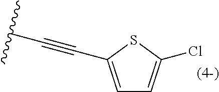 [SMILES: *C#Cc1ccc(Cl)s1] | 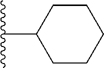 [SMILES: *C1CCCCC1] | 170 ± 12^{c}* | 1.48^{c} |
| 17 (MRS8247) | 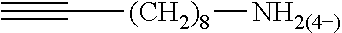 | 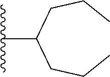 [SMILES: *C(CCC)CCC] | 206.8 ± 6.9 | 2.63 |
| 18 (MRS8255) | 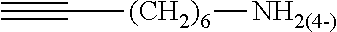 | 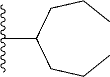 [SMILES: *C(CCC)CCC] | 190.27 | 0.630 |
| 19 (MRS8256) | 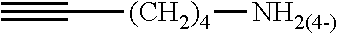 | 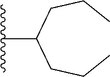 [SMILES: *C(CCC)CCC] | | |
| 20 (MRS8277) | 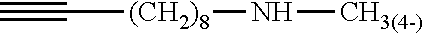 | 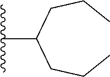 [SMILES: *C(CCC)CCC] | 224.5 (10 μM) | 0.328 |
| 21 (MRS8246) | 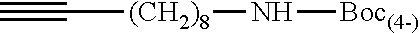 | 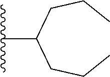 [SMILES: *C(CCC)CCC] | 136.7 ± 7.3 | 0.584 |
| 22 (MRS8253) | 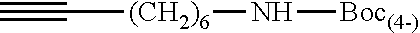 | 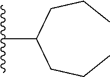 [SMILES: *C(CCC)CCC] | <200 | |
| 23 (MRS8254) | 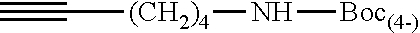 | 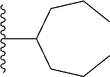 [SMILES: *C(CCC)CCC] | <200 | |
| 24 (MRS8280) | 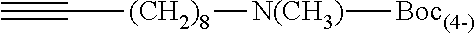 | 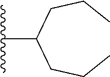 [SMILES: *C(CCC)CCC] | | |
| 25 (MRS8279) | 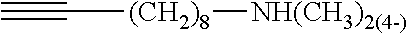 | 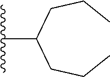 [SMILES: *C(CCC)CCC] | | |
| 26 (MRS8281) | 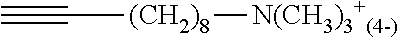 | 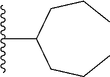 [SMILES: *C(CCC)CCC] | | |
| 27 (MRS8283) | 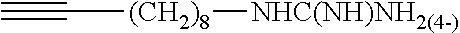 | 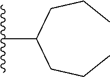 [SMILES: *C(CCC)CCC] | | |
| 28 (MRS8286) | 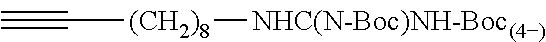 | 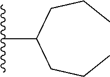 [SMILES: *C(CCC)CCC] | | |
| 29 (MRS8282) | 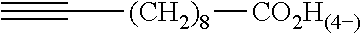 | 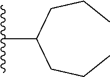 [SMILES: *C(CCC)CCC] | | |
| 30 (MRS8284) | 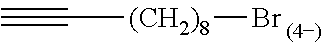 | 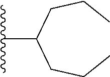 [SMILES: *C(CCC)CCC] | | |

NH-phenyl m-substituted derivatives

| | | |
|---|---|---|
| 31 (MRS8259) | 3-I | 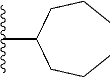 [SMILES: *C(CCC)CCC] |
| 32 (MRS8264) | 3-Cl-4-I | 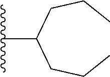 [SMILES: *C(CCC)CCC] |
| 33 (MRS8258) | 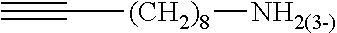 | 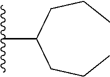 [SMILES: *C(CCC)CCC] |
| 34 (MRS8257) | 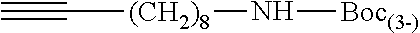 | 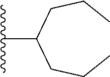 [SMILES: *C(CCC)CCC] |

^{a}Effect of PAM derivative (1.0 μM) on [³⁵S]GTPγS binding induced by Cl-IB-MECA 57 using WT hA₃ARs (mean ± SEM, n = 3). *P <0.05 (One-way ANOVA with Bonferroni-adjusted T-test for multiple comparisons) with respect to control in the absence of a PAM. ND, not determined.
^{b}Ratio of IC₅₀ values for Cl-IB-MECA 57 (DMSO control compared to 100 nM of each PAM derivative) in [³⁵S]GTPγS binding.
^{c}Data from Fallot et al., 2022.

TABLE 3
1H-Imidazo[4,5-c]quinolin-4-amine and Related Heterocyclic
Derivatives Synthesized and their Allosteric Effect on Agonist-Induced A₃AR Activation.
X = NH, unless noted otherwise.

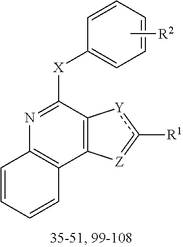

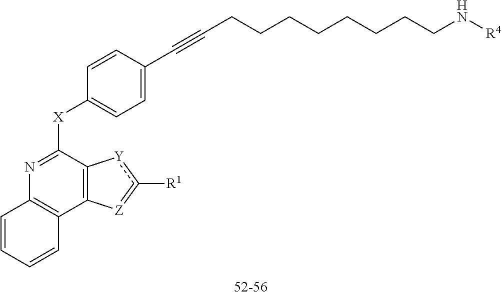

Imidazole-N-alkylated and 2-heptanyl phenyl ether and thioether derivatives

| | | | | | | IC₅₀ |
|---|---|---|---|---|---|---|
| Compound | R² or R⁴ | R¹ | Y | Z | Eₘₐₓª (%) | ratio^{b} |
| 35 (MRS8206) | 3,4-Cl₂ | 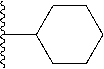 [SMILES: *C1CCCCC1] | N | N-CH₃ | 177.8 ± 11.5 | 1.12 |
| 36 (MRS8236) | 3,4-Cl₂ | 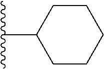 [SMILES: *C1CCCCC1] | N | N- CH₂CH₂OH | 119.3 ± 6.2 | 0.764 |
| 37 (MRS8278) | 4-I | 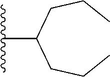 [SMILES: *C(CCC)CCC] | N | N-CH₂Ph | | |
| 38 (MRS8237) | H | 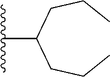 [SMILES: *C(CCC)CCC] | N (X = O) | NH | 131.7 ± 6.8 | 2.47 |
| 39 (MRS8238) | 3,4-Cl₂ | 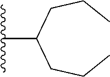 [SMILES: *C(CCC)CCC] | N (X = O) | NH | 167.2 ± 5.6 | 1.32 |
| 40 (MRS8242) | 3,4-Cl₂ | 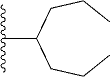 [SMILES: *C(CCC)CCC] | N (X = S) | NH | 149.9 ± 7.2 | 0.947 |

heteroatom replacement in core scaffold and other 2-cyclohexyl derivatives

| | | | | | | |
|---|---|---|---|---|---|---|
| 41 _{(RK005-} _{84-1, BS1002)} | 3,4-Cl₂ | 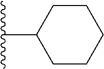 [SMILES: *C1CCCCC1] | N | O | 149.9 ± 8.8 | 0.406 |
| 42 _{(RK005-} _{79-1, BS1001)} | 3,4-Cl₂ | 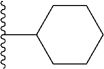 [SMILES: *C1CCCCC1] | N | S | 192.6 ± 11.2 | 0.505 |
| 43 _{(RK006-} _{62-1, BS1004)} | 3,4-Cl₂ | 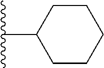 [SMILES: *C1CCCCC1] | O | NH | 176.9 ± 12.2 | 0.436 |
| 44 _{(RK006-} _{61-1, BS1003)} | 3,4-Cl₂ | 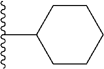 [SMILES: *C1CCCCC1] | S | NH | 187.4 ± 14.7 | 0.175 |
| 45 _{(RK006-} _{100-1,} _{BS1007)} | 3,4-Cl₂ | 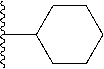 [SMILES: *C1CCCCC1] | N (X = NC H₃) | NH | 127.0 ± 6.0 | 0.491 |
| 46 _{(RK007-} _{4-1, BS1006)} | 3,4-Cl₂ | 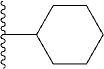 [SMILES: *C1CCCCC1] | N (X = O) | NH | 150.6 ± 5.8 | 0.738 |
| 47 _{(RK007-2-} _{1, BS1005)} | 3,4-Cl₂ | 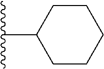 [SMILES: *C1CCCCC1] | N (X = S) | NH | 137.6 ± 7.3 | 1.11 |

other combined derivatives, including conjugates with reporter groups, such as
fluorophores

| | | | | |
|---|---|---|---|---|
| 48 | 4-I | 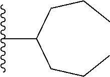 [SMILES: *C(CCC)CCC] | N | S |
| 49 | 4-I | 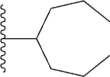 [SMILES: *C(CCC)CCC] | N | S |
| 50 | 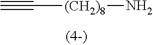 | 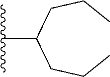 [SMILES: *C(CCC)CCC] | N | S |
| 51 | 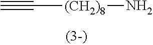 | 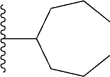 [SMILES: *C(CCC)CCC] | N | S |
| 52 | -(CH₂)₂-NH- AlexaFluor488 | 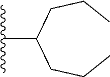 [SMILES: *C(CCC)CCC] | N | NH |
| 53 | -(CH₂)₂-NH- JaneliaFluor646 | 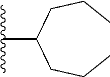 [SMILES: *C(CCC)CCC] | N | NH |
| 54 | -(CH₂)₂-NH-Cy5 | 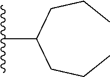 [SMILES: *C(CCC)CCC] | N | NH |
| 55 | -(CH₂)₂-NH-Cy7 | 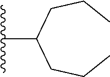 [SMILES: *C(CCC)CCC] | N | NH |
| 56 | -(CH₂)₂-NH- biotin | 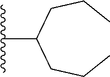 [SMILES: *C(CCC)CCC] | N | NH |

Other derivatives (Table 3 continued):

| | | | | |
|---|---|---|---|---|
| 99 | 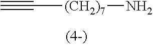 | 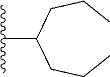 [SMILES: *C(CCC)CCC] | N | NH |
| 100 | 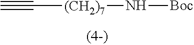 | 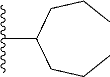 [SMILES: *C(CCC)CCC] | N | NH |
| 101 | 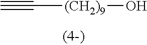 | 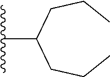 [SMILES: *C(CCC)CCC] | N | NH |
| 102 | 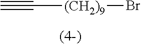 | 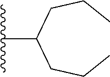 [SMILES: *C(CCC)CCC] | N | NH |
| 103 | 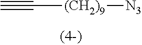 | 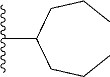 [SMILES: *C(CCC)CCC] | N | NH |
| 104 | 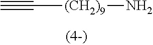 | 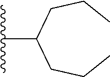 [SMILES: *C(CCC)CCC] | N | NH |
| 105 | 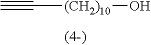 | 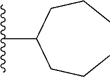 [SMILES: *C(CCC)CCC] | N | NH |
| 106 | 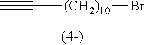 | 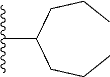 [SMILES: *C(CCC)CCC] | N | NH |
| 107 | 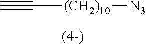 | 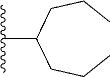 [SMILES: *C(CCC)CCC] | N | NH |
| 108 | 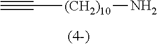 | 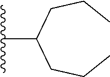 [SMILES: *C(CCC)CCC] | N | NH |

^{a}Effect of PAM derivative (1.0 μM) on [³⁵S]GTPγS binding induced by Cl-IB-MECA 57 using WT hA₃ARs (mean ± SEM, n = 3). *P <0.05 (One-way ANOVA with Bonferroni-adjusted T-test for multiple comparisons) with respect to control in the absence of a PAM. ND, not determined.
^{b}Ratio of IC₅₀ values for Cl-IB-MECA 57 (DMSO control compared to 100 nM of each PAM derivative) in [³⁵S]GTPγS binding.
^{c}Data from Fallot et al., 2022.

When comparing ethynyl-linked chains at the p-position of the phenyl ring, the longer terminal alkylamino derivative 17 increased both the Eₘₐₓ of the agonist and its potency, which was not found for previous A₃AR PAMs, which only displayed enhanced A₃AR efficacy. This potency enhancement was evident at 100 nM and 1 μM, illustrating that the more hydrophobic tethered ethynyl-linked octylamino chain of 17 was favored over the amide-linked 12. Curiously, when an amide bond was present in the extended chain in 12, or without a terminal amine in 13, the enhancement was much more modest, likely due to the incompatibility of the polar amide group with the hydrophobic lipid environment within the membrane bilayer. At concentrations of 100 nM and 1 μM 17, the agonist was significantly more potent than control (P=0.0114 and P<0.0001, respectively). The loss of enhancing activity of 17 at 10 UM is believed to be a function of the reduced aqueous solubility at this very high concentration. This enhancement by 17 contrasts with the shorter chain hexyl-amino homologue 16. However, there was no left-shift of the activation curve by 16 at concentrations of 100 nM and 1 μM. The N-Boc derivative of 17, i.e., 21, displayed minimal allosteric enhancement, suggesting that a terminal amino group of 17, which would be largely positively charged at physiological pH, is needed for this exceptional activity. The Boc-protected analogue 17 was much less efficacious except at the highest (10 μM) concentration, at which concentration a right-shift was present, indicative of potential receptor antagonism.

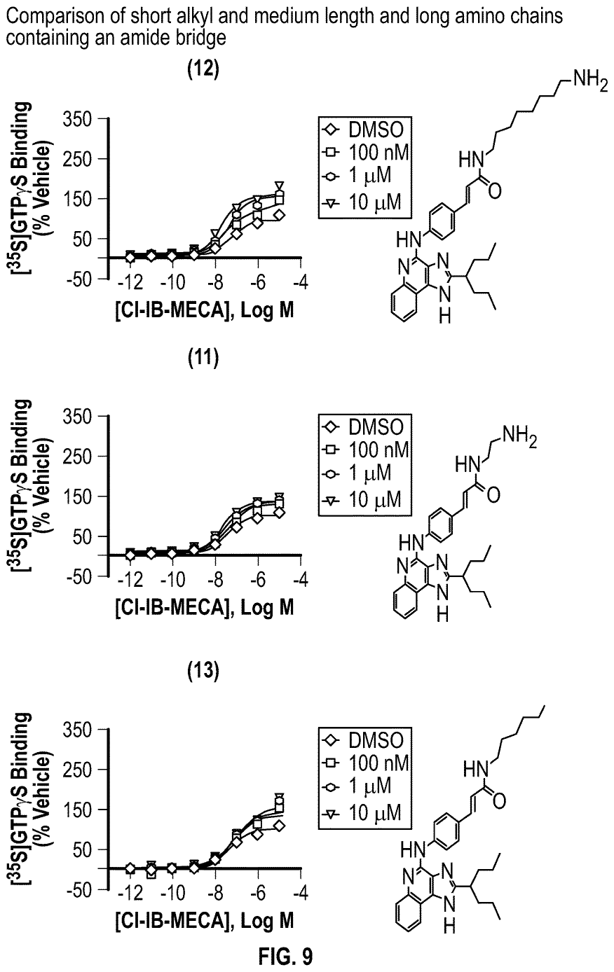

FIG. 9 depicts the results of determination of EC₅₀ and Eₘₐₓ of agonist Cl-IB-MECA in [³⁵S]GTPγS binding assays in the presence of increasing concentrations of three derivatives, 12, 11, and 13, containing a p-acryloyl group on the 4-phenylamino substituent. The modest Eₘₐₓ increase induced by these three derivatives is indicative of hA3AR allosteric enhancement.

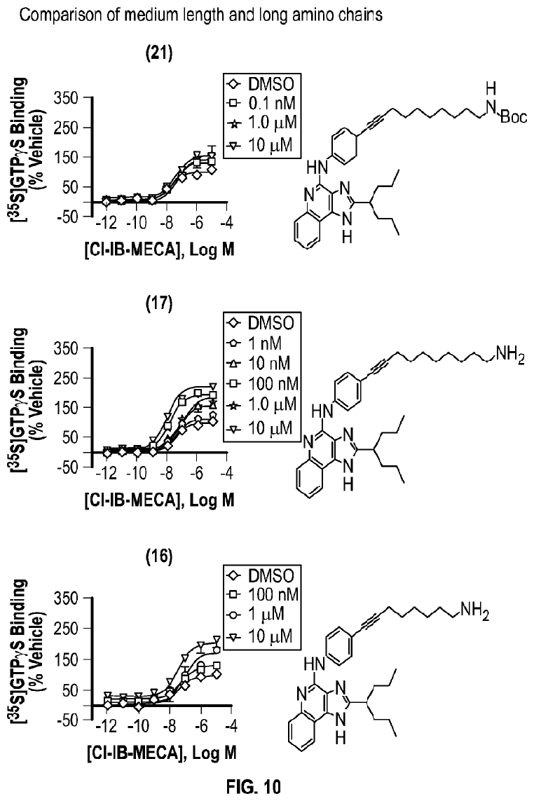

FIG. 10 depicts the results of determination of EC₅₀ and Eₘₐₓ of agonist Cl-IB-MECA in [³⁵S]GTPγS binding assays in the presence of increasing concentrations of three derivatives, 21, 17 and 16, containing a p-alkynyl group on the 4-phenylamino substituent. The substantial Eₘₐₓ increase by 16 and 17 indicates hA3AR allosteric enhancement. The left-shift of the activation curve by 17 (100 nM and 1 μM) to a more potent EC₅₀ is indicative of a qualitatively different mechanism of hA3AR allosteric enhancement compared to other PAM derivatives. The loss of the enhancement at the highest concentration (10 μM) is ascribed to limited aqueous solubility. The Boc-protected analogue 21 was much less efficacious in increasing Eₘₐₓ and had no effect on EC₅₀. At the highest (10 μM) concentration, a right-shift was present, indicative of receptor negative modulation.

### EC₅₀ of Cl-IB-MECA

100 nM significantly more potent than control P=0.0114
1 μM significantly more potent than control P<0.0001
10 μM significantly less potent than control P=0.0015

### Eₘₐₓ of Cl-IB-MECA, for MRS8247, N=4-5

10 nM significantly greater than control P<0.0001
100 nM significantly greater than control P<0.0001
1 μM significantly greater than control P<0.0001
10 μM significantly greater than control P<0.0001

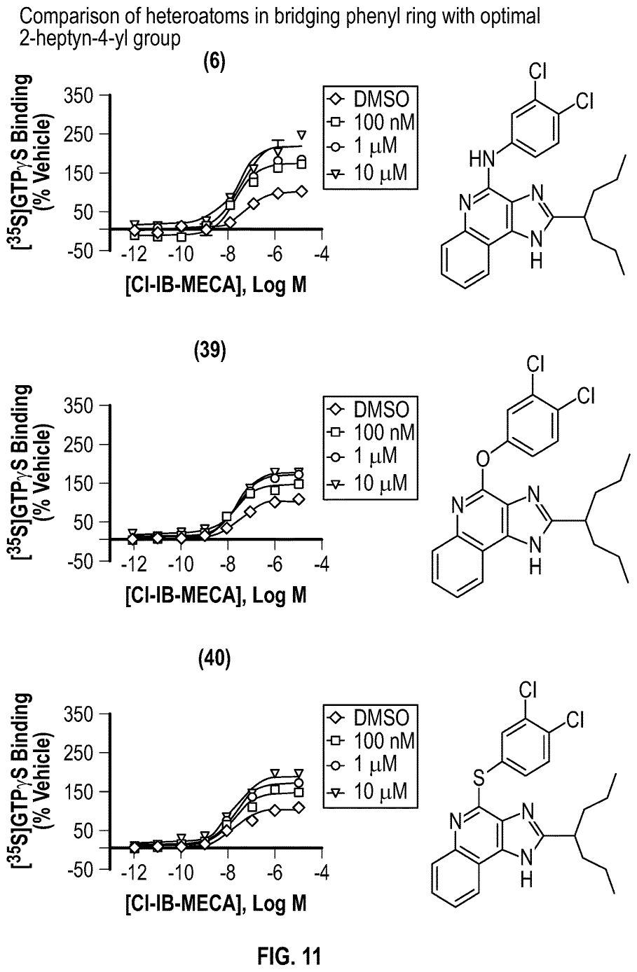

FIG. 11 depicts the results of determination of EC₅₀ and Eₘₐₓ of agonist CI-IB-MECA in [³⁵S]GTPγS binding assays in the presence of increasing concentrations of three compounds, 6, 39, and 40, containing N, O, or S atoms bridging the 4-heteroatom-phenyl substituent. The more modest Eₘₐₓ increase induced by 39 and 40, compared to reference compound 6 is indicative of a reduction of hA₃AR allosteric enhancement.

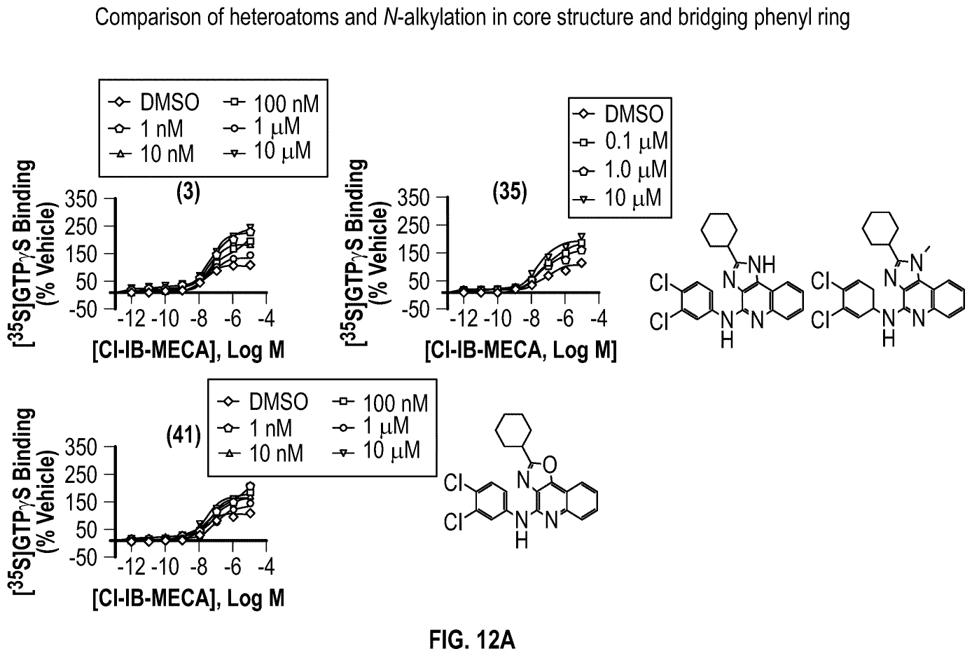

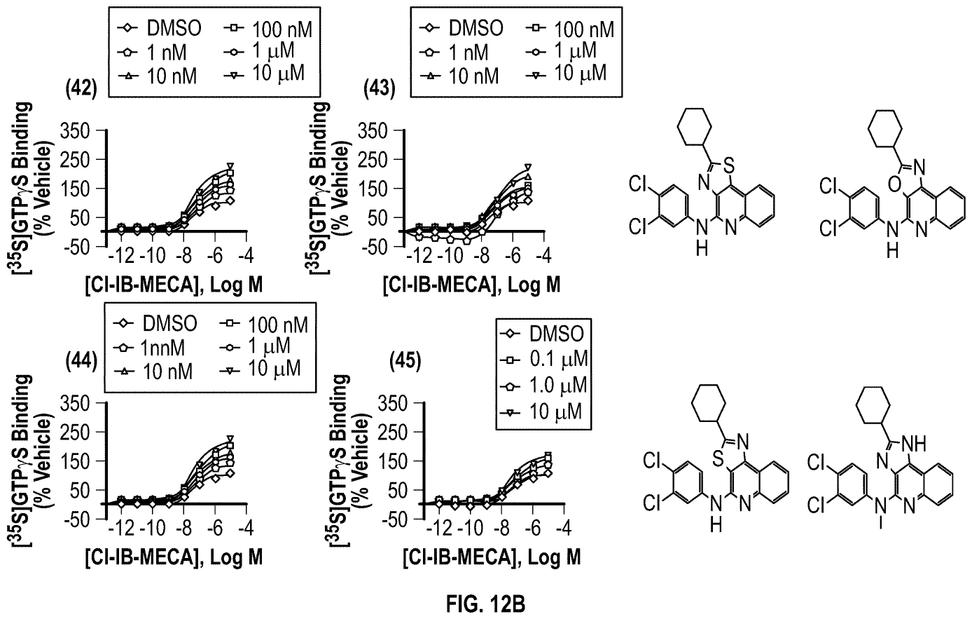

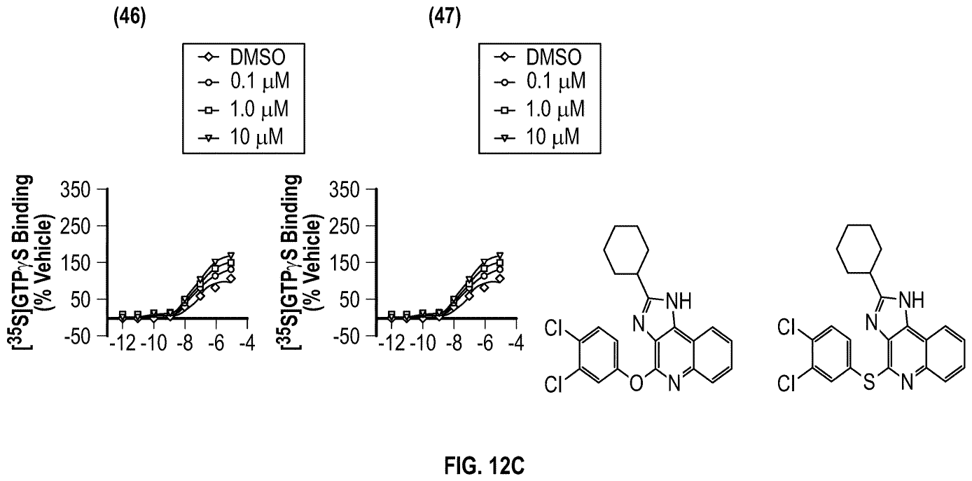

FIG. 12 depicts the results of determination of EC₅₀ and Eₘₐₓ of agonist Cl-IB-MECA in [³⁵S]GTPγS binding assays in the presence of increasing concentrations of eight derivatives, 35 and 41-47, compared to reference compound 3, all of which contain 2-cyclohexyl and 3,4-dichlorophenyl groups. The changes include NH, N-CH₃, O, or S groups bridging the 4-heteroatom-3,4-dichlorophenyl substituent. The smaller Eₘₐₓ increase by 41 and 45-47, compared to 3 as well as 42-44, is indicative of greatly reduced hA₃AR allosteric enhancement, suggesting that the 4—NH is important for recognition at the hA₃AR allosteric binding site.

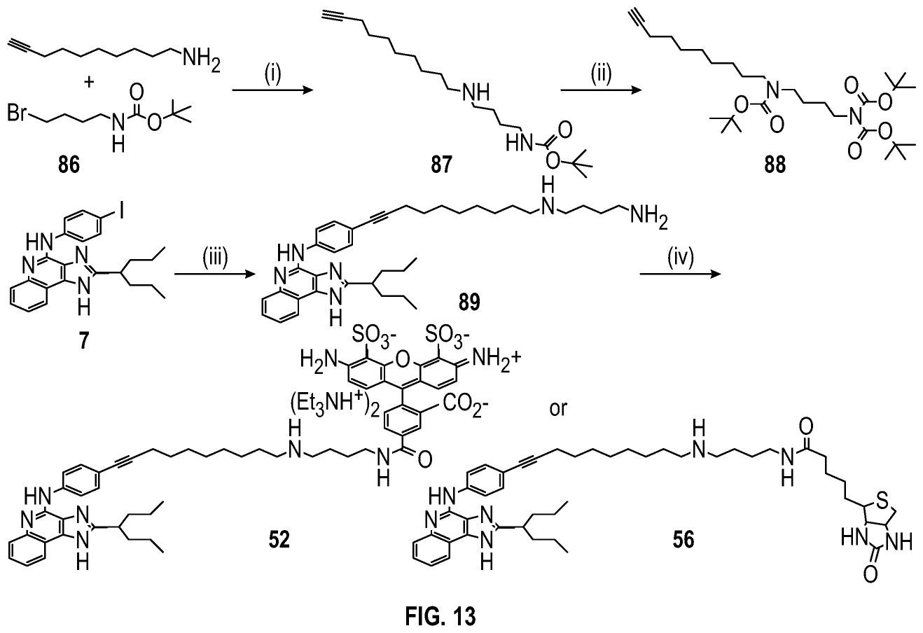

FIG. 13 depicts a synthesis of PAM derivatives with reporter groups as pharmacological tool compounds. Compounds 52-55 are fluorescent analogues of 17. The amino group on the chain of the new analogue is designed to retain its positive charge to enable it to interact with the membrane polar head groups on the cytosolic side of the membrane. The fluorophore would likely be in a polar environment of the cytosol.

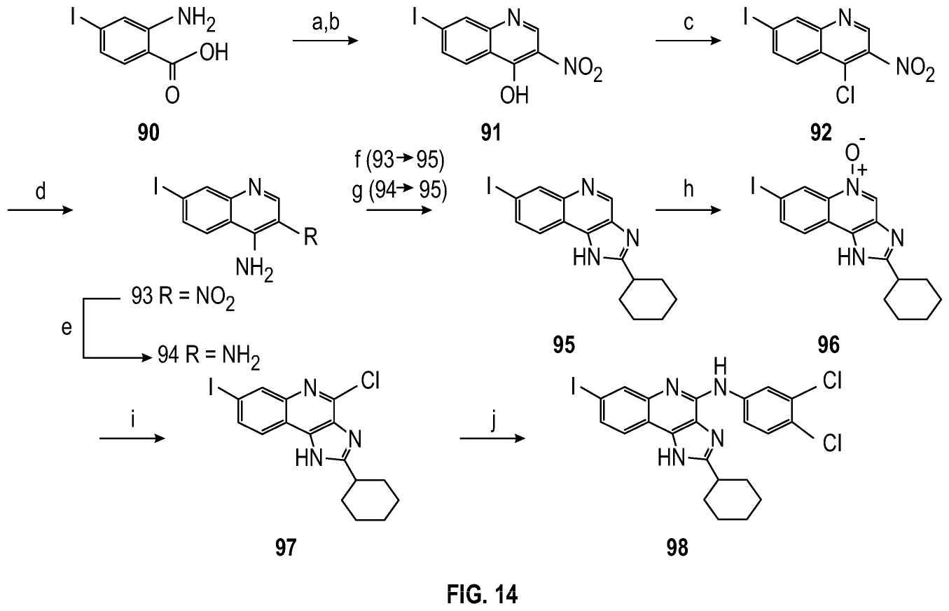

FIG. 14 depicts a reaction scheme to synthesize compound 98 in accordance with an aspect of the invention.

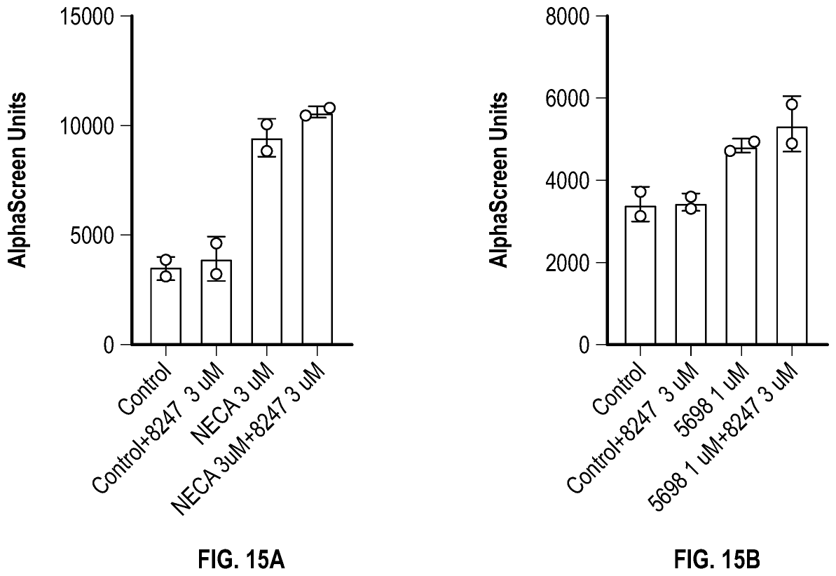

FIG. 15 depicts the results of determination of the modulation of EC₅₀ and Eₘₐₓ of agonist Cl-IB-MECA in [³⁵S]GTPγS functional assays and in agonist radioligand binding, indicative that the hA₃AR PAM binding site is located in TM7 and helix 8, with the possible secondary involvement of TM1.

### EXAMPLE 4

This example shows the result of a modeling study which predicted the binding site. A model of A₃AR structure in the active state was generated by homology modelling using an active-state cryo-EM structure of A₁AR (PDB ID: 7LD4) (51) as template. The model was obtained using the Prime (52, 53) tool from the Schrödinger suite (54), and prepared with the Protein Preparation Wizard (55) tool. Non-conserved residues were minimized with the OPLS4 (56) force field.

The SiteMap (57, 58) tool was employed for the prediction of the binding site on the receptor surface. The top ranked predicted binding site corresponded to the A₃AR orthosteric pocket, so it was not considered. The second ranked site, which is located at the interface among TM1, TM7 and helix 8, was then selected as putative allosteric pocket.

LUF6000 was docked at the predicted binding pocket using an Induced Fit (59, 60, 61) docking procedure. For the initial docking stage, a grid centered on Val33, with an inner box of 10 Å and an outer box of 30 Å, was adopted. Residues within 5 Å from the ligand were refined, and the XP scoring function was adopted for the redocking phase. The first ranked pose was selected as putative binding mode of the ligand.

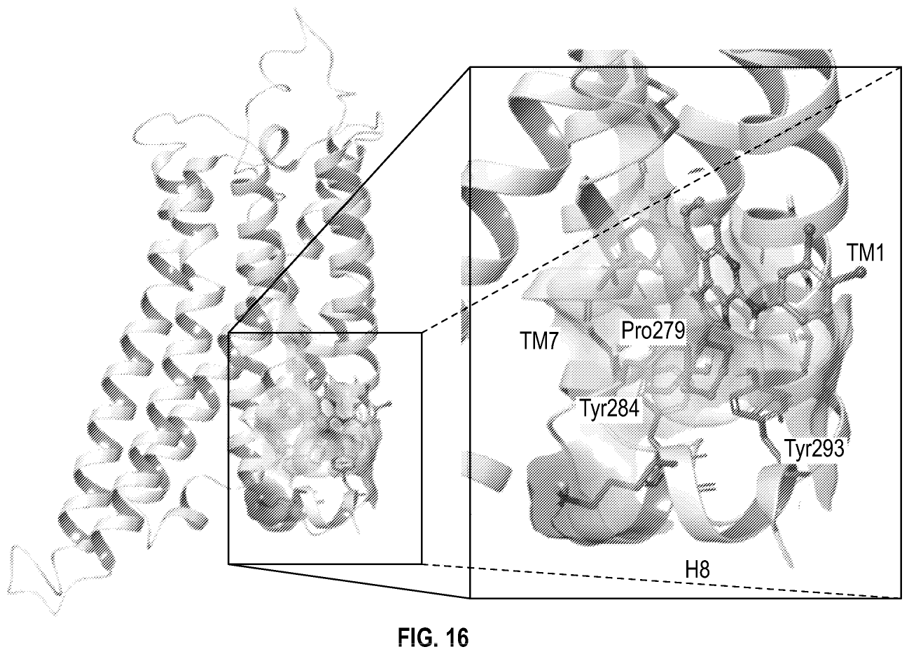

FIG. 16 depicts a result of molecular modeling, showing the binding site of the compounds in accordance with an aspect of the invention.

### EXAMPLE 5

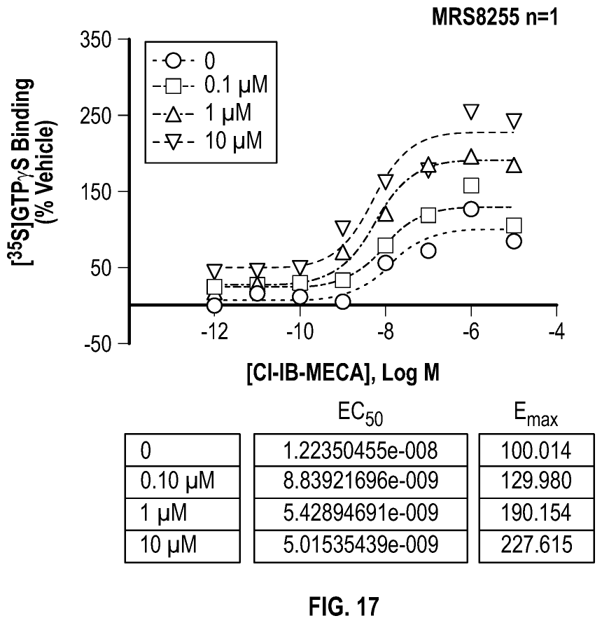

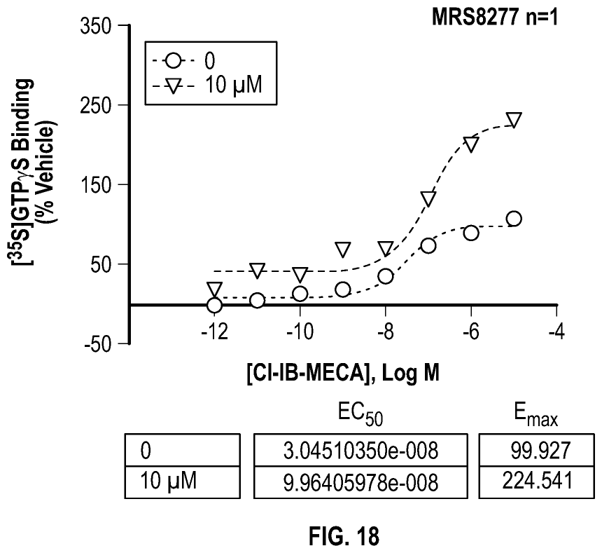

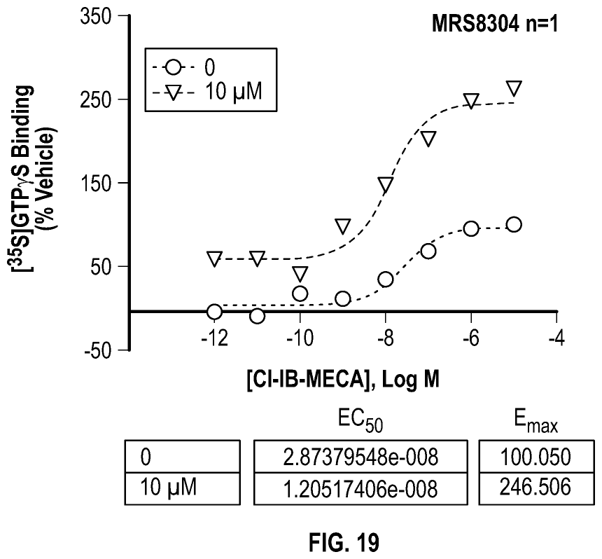

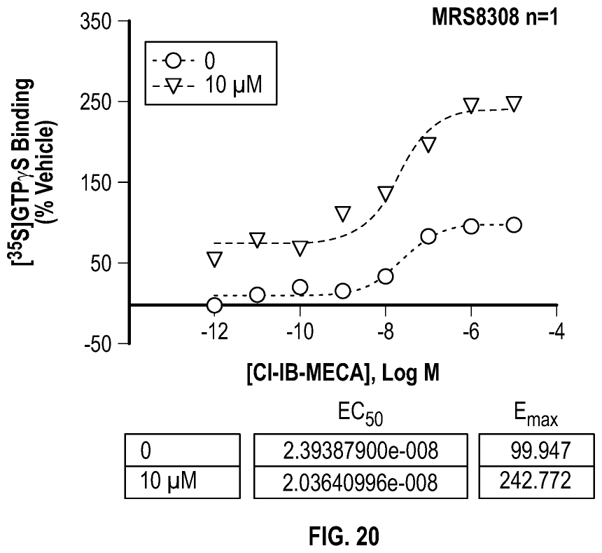

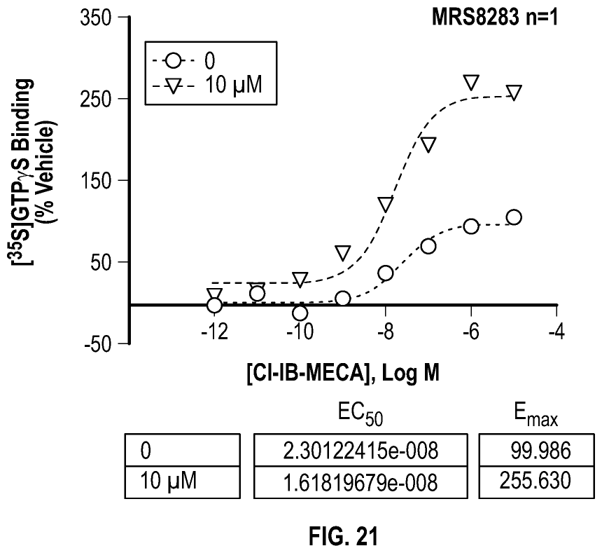

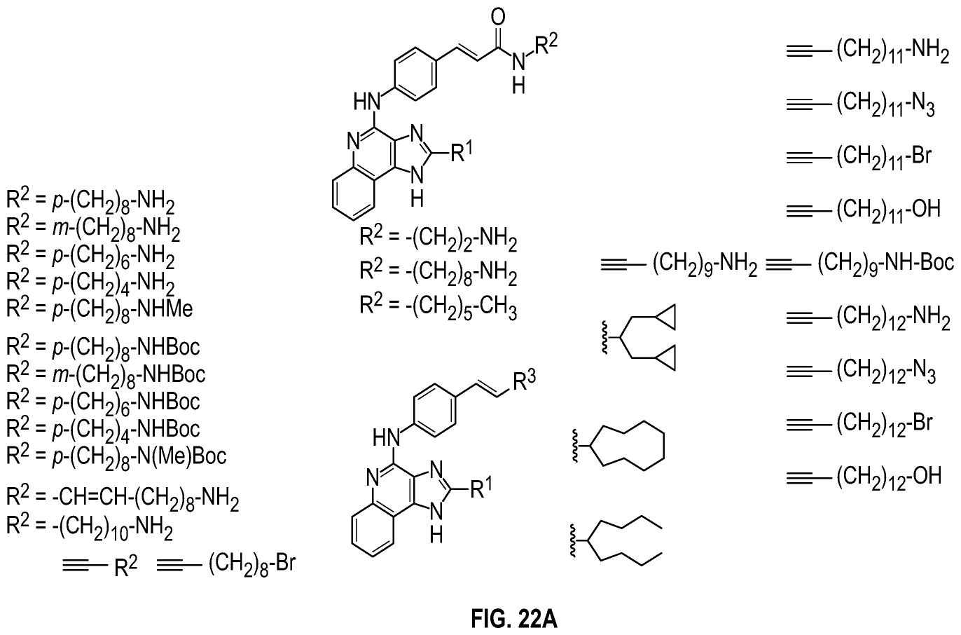

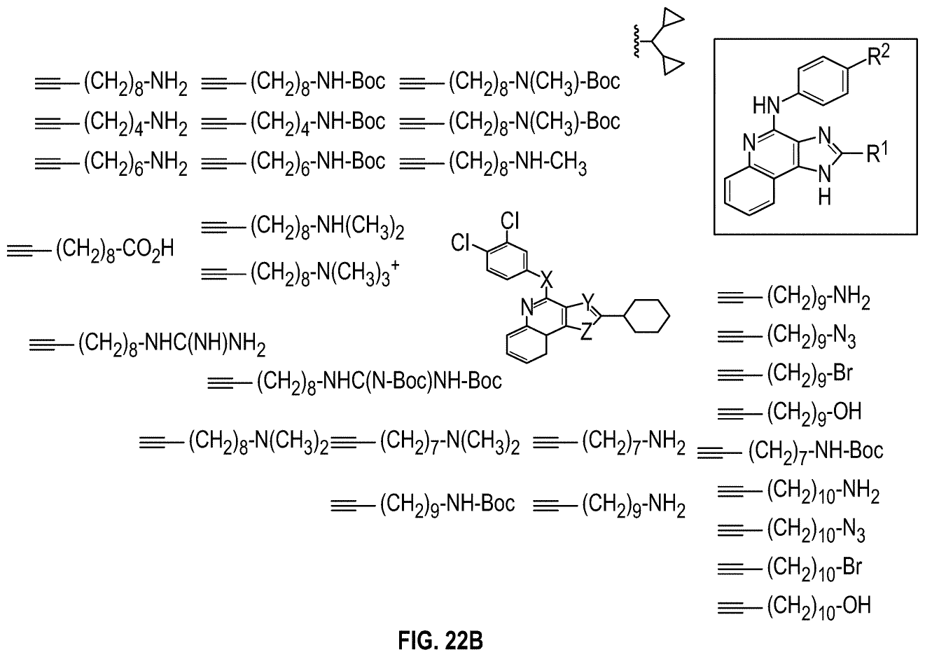

This example illustrates the biological activity of the compounds in accordance with an aspect of the invention. See FIG. 17-21.

### EXAMPLE 6

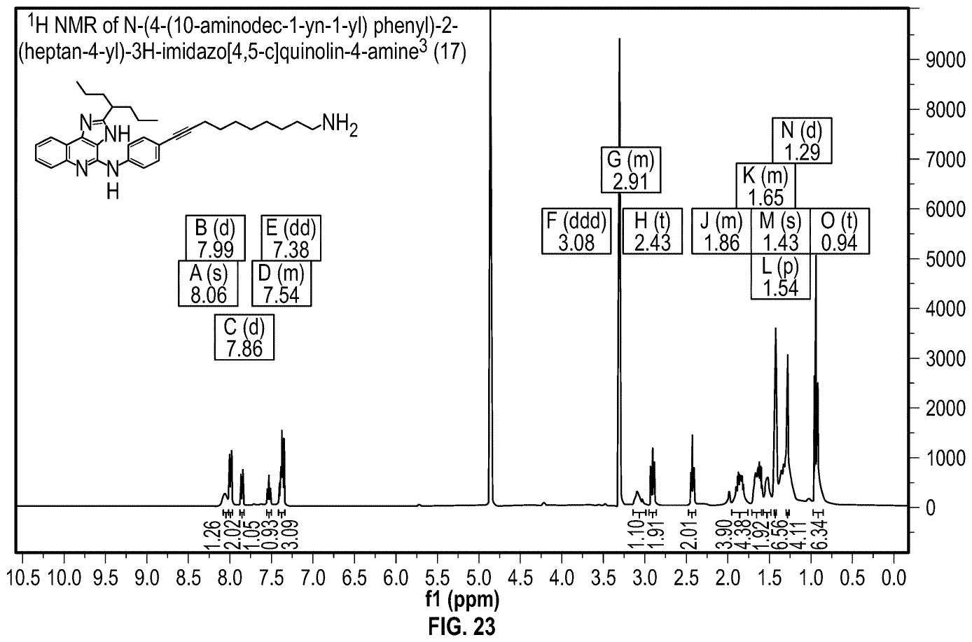

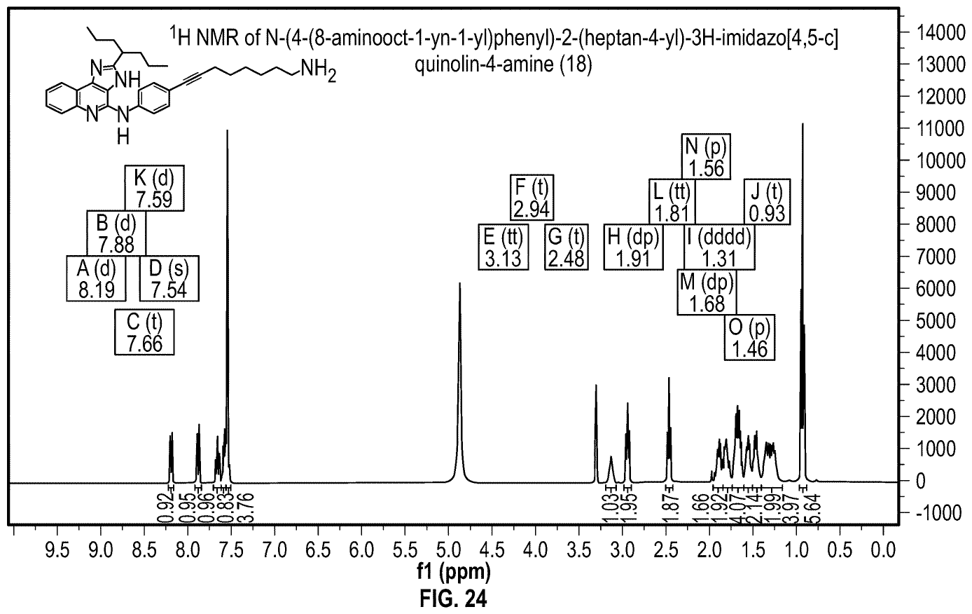

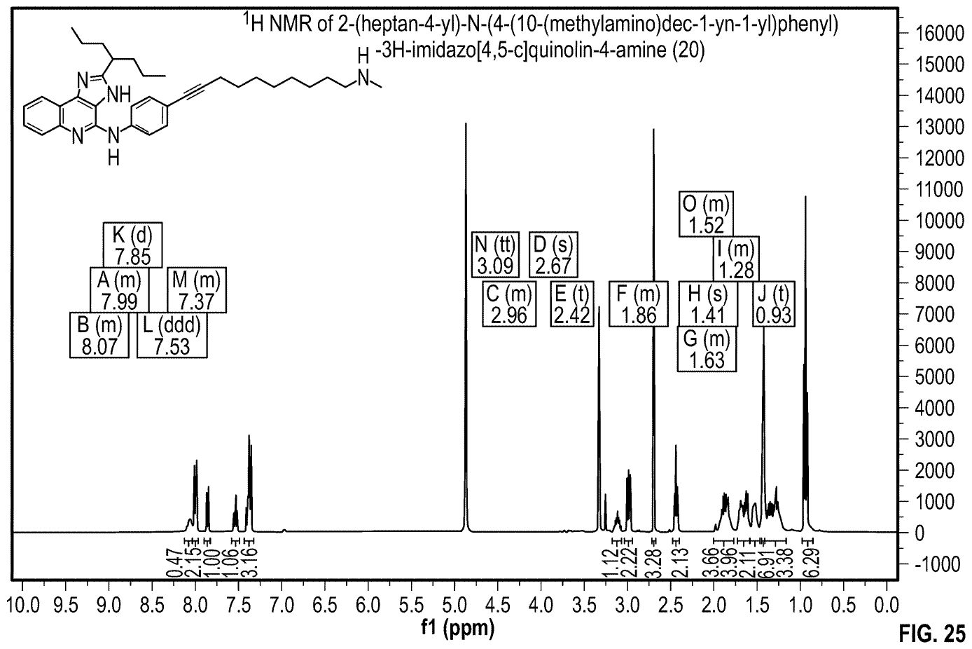

FIG. 23 depicts the NMR results obtained on compound 17, FIG. 24 depicts the NMR results obtained on another compound whose structural formula is shown, in accordance with an aspect of the invention, and FIG. 25 depicts the NMR results obtained on compound 20, in accordance with an aspect of the invention.

### REFERENCES

(1) Gessi, S.; Merighi, S.; Varani, K. Adenosine receptors: The status of the art. In The Adenosine Receptors, Borea, P. A., Varani, K., Gessi, S., Merighi, S., Vincenzi, F. Eds.; Springer International Publishing, 2018; pp 1-11.
(2) Linden, J. Adenosine in tissue protection and tissue regeneration. Mol. Pharmacol. 2005, 67 (5), 1385. DOI: 10.1124/mol.105.011783.
(3) Borea, P. A.; Varani, K.; Vincenzi, F.; Baraldi, P. G.; Tabrizi, M. A.; Merighi, S.; Gessi, S. The A₃ adenosine receptor: History and perspectives. Pharmacol. Rev. 2015, 67 (1), 74. DOI: 10.1124/pr. 113.008540.
(4) Jacobson, K. A.; Tosh, D. K.; Jain, S.; Gao, Z.-G. Historical and current adenosine receptor agonists in preclinical and clinical development. Front. Cell. Neurosci. 2019, 13 (124), Review. DOI: 10.3389/fncel.2019.00124.
(5) Changeux, J.-P.; Edelstein, S. J. Allosteric mechanisms of signal transduction. Science 2005, 308 (5727), 1424-1428. DOI: doi: 10.1126/science.1108595.
(6) Draper-Joyce, C. J.; Bhola, R.; Wang, J.; Bhattarai, A.; Nguyen, A. T. N.; Cowie-Kent, I.; O'Sullivan, K.; Chia, L. Y.; Venugopal, H.; Valant, C.; Thal, D. M., Wootten, D.; Panel, N., Carlsson, J.; Christie, M. J., White, P. J., Scammells, P., May, L. T., Sexton, P. M.; Danev, R., Miao, Y., Glukhova, A.; Imlach, W. L.; Christopoulos, A. Positive allosteric mechanisms of adenosine A₁ receptor-mediated analgesia. Nature 2021, 597 (7877), 571-576. DOI: 10.1038/s41586-021-03897-2.
(7) Conn, P. J.; Christopoulos, A.; Lindsley, C. W. Allosteric modulators of GPCRs: a novel approach for the treatment of CNS disorders. Nat. Rev. Drug. Discov. 2009, 8 (1), 41-54. DOI: 10.1038/nrd2760.
(8) Merighi, S.; Simioni, C.; Lane, R.; IJzerman, A. P. Regulation of second messenger systems and intracellular pathways. In A₃ Adenosine Receptors from Cell Biology to Pharmacology and Therapeutics, Borea, P. A. Ed.; Springer Netherlands, 2010; pp 61-73.
(9) Foster, D. J.; Conn, P. J. Allosteric modulation of GPCRs: New insights and potential utility for treatment of schizophrenia and other CNS disorders. Neuron 2017, 94 (3), 431-446. DOI: https://doi.org/10.1016/j.neuron.2017.03.016.
(10) Schaddelee, M. P.; Read, K. D.; Cleypool, C. G.; IJzerman, A. P.; Danhof, M.; de Boer, A. G. Brain penetration of synthetic adenosine A₁ receptor agonists in situ: role of the rENT1 nucleoside transporter and binding to blood constituents. Eur. J. Pharm. Sci. 2005, 24 (1), 59-66. DOI: 10.1016/j.ejps.2004.09.010.
(11) Liston, T. E.; Hinz, S.; Müller, C. E.; Holstein, D. M.; Wendling, J.; Melton, R. J.; Campbell, M.; Korinek, W. S.; Suresh, R. R.; Sethre-Hofstad, D. A.; Gao, Z. G.; Tosh, D. K.; Jacobson, K. A.; Lechleiter, J. D. Nucleotide P₂Y₁ receptor agonists are in vitro and in vivo prodrugs of A₁/A₃ adenosine receptor agonists: implications for roles of P₂Y₁ and A₁/A₃ receptors in physiology and pathology. Purinergic Signal. 2020, 16 (4), 543-559. DOI: 10.1007/s11302-020-09732-z.
(12) Fredholm, B. B.; IJzerman, A. P.; Jacobson, K. A.; Linden, J.; Müller, C. E. International Union of Basic and Clinical Pharmacology. LXXXI. Nomenclature and classification of adenosine receptors—an update. Pharmacol. Rev. 2011, 63 (1), 1-34. DOI: 10.1124/pr.110.003285.
(13) Jacobson, K. A.; Gao, Z.-G. Chapter 11 Allosteric modulators of adenosine, P2Y and P2X receptors. In Allosterism in Drug Discovery, The Royal Society of Chemistry, 2017; pp 247-270.
(14) Slosky, L. M.; Caron, M. G.; Barak, L. S. Biased allosteric modulators: New frontiers in GPCR drug discovery. Trends Pharmacol. Sci. 2021, 42 (4), 283-299. DOI: 10.1016/j.tips.2020.12.005.
(15) Gao, Z.-G.; Melman, N.; Erdmann, A.; Kim, S. G.; Müller, C. E.; IJzerman, A. P.; Jacobson, K. A. Differential allosteric modulation by amiloride analogues of agonist and antagonist binding at A₁ and A₃ adenosine receptors. Biochem. Pharmacol. 2003, 65 (4), 525-534. DOI: https://doi.org/10.1016/S0006-2952 (02) 01556-3.
(16) Gao, Z.-G.; Van Muijlwijk-Koezen, J. E.; Chen, A.; Müller, C. E.; IJzerman, A. P.; Jacobson, K. A. Allosteric modulation of A₃ adenosine receptors by a series of 3-(2-pyridinyl) isoquinoline derivatives. Mol. Pharmacol. 2001, 60 (5), 1057. DOI: 10.1124/mol.60.5.1057.
(17) Gao, Z.-G.; Kim, S. G.; Soltysiak, K. A.; Melman, N.; IJzerman, A. P.; Jacobson, K. A. Selective allosteric enhancement of agonist binding and function at human A₃ adenosine receptors by a series of imidazoquinoline derivatives. Mol. Pharmacol. 2002, 62 (1), 81-89. DOI: 10.1124/mol.62.1.81.
(18) Göblyös, A.; Gao, Z.-G.; Brussee, J.; Connestari, R.; Santiago, S. N.; Ye, K.; IJzerman, A. P.; Jacobson, K. A. Structure-activity relationships of new ¹H-imidazo[4,5-c] quinolin-4-amine derivatives as allosteric enhancers of the A₃ adenosine receptor. J. Med. Chem. 2006, 49 (11), 3354-3361. DOI: 10.1021/jm060086s.
(19) Kim, Y.; de Castro, S.; Gao, Z.-G.; IJzerman, A. P.; Jacobson, K. A. Novel 2— and 4-substituted 1H-imidazo[4,5-c]quinolin-4-amine derivatives as allosteric modulators of the A₃ adenosine receptor. J. Med. Chem. 2009, 52 (7), 2098-2108. DOI: 10.1021/jm801659w.
(20) Heitman, L. H.; Göblyös, A.; Zweemer, A. M.; Bakker, R.; Mulder-Krieger, T.; van Veldhoven, J. P. D.; de Vries, H.; Brussee, J.; IJzerman, A. P. A series of 2,4-disubstituted quinolines as a new class of allosteric enhancers of the adenosine A₃ receptor. J. Med. Chem. 2009, 52 (4), 926-931. DOI: 10.1021/jm8014052.
(21) Gao, Z.-G.; Verzijl, D.; Zweemer, A.; Ye, K.; Göblyös, A.; IJzerman, A. P.; Jacobson, K. A. Functionally biased modulation of A₃ adenosine receptor agonist efficacy and potency by imidazoquinolinamine allosteric enhancers. Biochem. Pharmacol. 2011, 82 (6), 658-668. DOI: 10.1016/j.bcp.2011.06.017.
(22) Fisher, C. L.; Fallot, L. B.; Wan, T. C.; Keyes, R. F.; Suresh, R. R.; Rothwell, A. C.; Gao, Z. G.; Smith, B. C.; Jacobson, K. A.; Auchampach, J. A. Structure activity relationship of dual action purine nucleoside allosteric modulators of the A₃ adenosine receptor, in preparation. ACS Pharmacol. Transl. Sci. 2022, submitted.
(23) Gao, D.; Xiao, Q.; Zhang, M.; Li, Y. Design, synthesis and biological evaluation of benzyloxyphenyl-methylaminophenol derivatives as STAT3 signaling pathway inhibitors. Bioorg. Med. Chem. 2016, 24 (11), 2549-2558. DOI: https://doi.org/10.1016/j.bmc.2016.04.022.
(24) Beutner, G. L.; Young, I. S.; Davies, M. L.; Hickey, M. R.; Park, H.; Stevens, J. M.; Ye, Q. TCFH-NMI: Direct access to N-acyl imidazoliums for challenging amide bond formations. Org. Lett. 2018, 20 (14), 4218-4222. DOI: 10.1021/acs.orglett.8b01591.
(25) Ruiz-Castillo, P.; Buchwald, S. L. Applications of palladium-catalyzed C—N cross-coupling reactions. Chem. Rev. 2016, 116 (19), 12564-12649. DOI: 10.1021/acs.chemrev.6b00512.
(26) Ghodke, P. P.; Pradeepkumar, P. I. Chapter 10-Nucleoside modification using Buchwald-Hartwig amination reactions. In Palladium-Catalyzed Modification of Nucleosides, Nucleotides and Oligonucleotides, Kapdi, A. R., Maiti, D., Sanghvi, Y. S. Eds.; Elsevier, 2018; pp 295-333.
(27) Fors, B. P.; Krattiger, P.; Strieter, E.; Buchwald, S. L. Water-mediated catalyst preactivation: An efficient protocol for C—N cross-coupling reactions. Org. Lett. 2008, 10 (16), 3505-3508. DOI: 10.1021/01801285g.
(28) Meanwell, N. A. Fluorine and fluorinated motifs in the design and application of bioisosteres for drug design. J. Med. Chem. 2018, 61 (14), 5822-5880. DOI: 10.1021/acs.jmedchem.7b01788.
(29) Miller, M. A.; Sletten, E. M. A general approach to biocompatible branched fluorous tags for increased solubility in perfluorocarbon solvents. Org. Lett. 2018, 20 (21), 6850-6854. DOI: 10.1021/acs.orglett.8b02976.
(30) Kouji, Y.; Mikiro, Y. Novel fluorine-containing compound, its preparation and antiepileptic agent. JPH0421652A, Jan. 24, 1992.
(31) Denmark, S. E.; Edwards, J. P. A comparison of (chloromethyl)- and (iodomethyl) zinc cyclopropanation reagents. J. Org. Chem. 1991, 56 (25), 6974-6981. DOI: 10.1021/jo00025a007.
(32) Singh, V.; Tosh, D. K.; Mobin, S. M. Synthesis of embellished bicyclo[2.2.2]octenones and a sigmatropic 1,2-acyl shift in an excited state: a novel and stereoselective route to (±)-hirsutic acid C and complicatic acid. Tet. Lett. 2004, 45 (8), 1729-1732. DOI: https://doi.org/10.1016/j.tetlet.2003.12.095
(33) Singh, V.; Pal, S.; Tosh, D. K.; Mobin, S. M. Molecular complexity from aromatics. Cycloaddition of cyclohexa-2,4-dienones, sigmatropic 1,2-acyl shift and ring-closing metathesis: a new, efficient, and stereoselective synthesis of (±)-hirsutic acid C and medium ring carbocycles. Tetrahedron 2007, 63 (11), 2446-2454. DOI: https://doi.org/10.1016/j.tet.2007.01.006.
(34) Appendino, G.; Spagliardi, P.; Sterner, O.; Milligan, S. Structure-activity relationships of the estrogenic sesquiterpene ester ferutinin. modification of the terpenoid core. J. Nat. Prod. 2004, 67 (9), 1557-1564. DOI: 10.1021/np049796w.
(35) Dikošová, L.; Laceková, J.; Záborský, O.; Fischer, R. Synthesis of 3-substituted isoxazolidin-4-ols using hydroboration-oxidation reactions of 4,5-unsubstituted 2,3-dihydroisoxazoles. Beilstein J. Org. Chem. 2020, 16, 1313-1319. DOI: 10.3762/bjoc.16.112.
(36) Beghetto, V.; Scrivanti, A.; Bertoldini, M.; Aversa, M.; Zancanaro, A.; Matteoli, U. A Practical, enantioselective synthesis of the fragrances canthoxal and silvial®, and evaluation of their olfactory activity. Synthesis 2015, 47 (02), 272-288.
(37) Qadir, M.; Möchel, T.; Hii, K. K. Examination of ligand effects in the Heck arylation reaction. Tetrahedron 2000, 56 (40), 7975-7979. DOI: https://doi.org/10.1016/S0040-4020 (00) 00703-1.
(38) Thorand, S.; Krause, N. Improved procedures for the palladium-catalyzed coupling of terminal alkynes with aryl bromides (Sonogashira coupling). J. Org. Chem. 1998, 63 (23), 8551-8553. DOI: 10.1021/jo9808021.
(39) Sonogashira, K. Development of Pd-Cu catalyzed cross-coupling of terminal acetylenes with sp2-carbon halides. J. Organomet. Chem. 2002, 653 (1), 46-49. DOI: https://doi.org/10.1016/S0022-328X (02) 01158-0.
(40) Sloan, N. L.; Luthra, S. K.; McRobbie, G.; Pimlott, S. L.; Sutherland, A. A one-pot radioiodination of aryl amines via stable diazonium salts: preparation of ¹²⁵I-imaging agents. Chem. Comm. 2017, 53 (80), 11008-11011, 10.1039/C7CC06211G. DOI: 10.1039/C7CC06211G.
(41) Besnard, J.; Ruda, G. F.; Setola, V.; Abecassis, K.; Rodriguiz, R. M.; Huang, X.-P.; Norval, S.; Sassano, M. F.; Shin, A. I.; Webster, L. A.; Simeons, F. R.; Stojanovski, L.; Prat, A.; Seidah, N. G.; Constam, D. B.; Bickerton, G. R.; Read, K. D.; Wetsel, W. C.; Gilbert, I. H.; Roth, B. L.; Hopkins, A. L. Automated design of ligands to polypharmacological profiles. Nature 2012, 492 (7428), 215-220. DOI: 10.1038/nature11691.
(42) Tosh, D. K.; Salmaso, V.; Campbell, R. G.; Rao, H.; Bitant, A.; Pottie, E.; Stove, C. P.; Liu, N.; Gavrilova, O.; Gao, Z. G.; Auchampach, J. A.; Jacobson, K. A. A₃ adenosine receptor agonists containing dopamine moieties for enhanced interspecies affinity. Eur. J. Med. Chem. 2022, 228, 113983. DOI: 10.1016/j.ejmech.2021.113983.
(43) Ciancetta, A.; Jacobson, K. A. Structural probing and molecular modeling of the A₃ adenosine receptor: A focus on agonist binding. Molecules 2017, 22 (3). DOI: 10.3390/molecules22030449.
(44) Ouvry, G. Recent applications of seven-membered rings in drug design. Bioorg. Med. Chem. 2022, 57, 116650. DOI: https://doi.org/10.1016/j.bmc.2022.116650.
(45) Sato, J.; Makita, N.; Iiri, T. Inverse agonism: the classic concept of GPCRs revisited. Endocr. J. 2016, 63 (6), 507-514. DOI: 10.1507/endocrj.EJ16-0084.
(46) Christopoulos, A.; Changeux, J. P.; Catterall, W. A.; Fabbro, D.; Burris, T. P.; Cidlowski, J. A.; Olsen, R. W.; Peters, J. A.; Neubig, R. R.; Pin, J. P.; Sexton, P. M.; Kenakin, T. P.; Ehlert, F. J.; Spedding, M.; Langmead, C. J. International Union of Basic and Clinical Pharmacology. XC. Multisite pharmacology: recommendations for the nomenclature of receptor allosterism and allosteric ligands. Pharmacol. Rev. 2014, 66 (4), 918-947. DOI: 10.1124/pr.114.008862.
(47) Sekhar, T.; Thriveni, P.; Venkateswarlu, A.; Daveedu, T.; Peddanna, K.; Sainath, S. B. One-pot synthesis of thiazolo[3,2-a]pyrimidine derivatives, their cytotoxic evaluation and molecular docking studies. Spectrochim. Acta Part A: Mol. Biomol. Spectr. 2020, 231, 118056. DOI: https://doi.org/10.1016/j.saa.2020.118056.
(48) Tsuchida, N.; Yamazaki, S.; Yamabe, S. A new mechanism for the Favorskii rearrangement. Org. Biomol. Chem. 2008, 6 (17), 3109-3117, 10.1039/B806577B. DOI: 10.1039/B806577B.
(49) Suresh, R. R.; Gao, Z. G.; Salmaso, V.; Chen, E.; Campbell, R. G.; Poe, R. B.; Liston, T. E.; Jacobson, K. A. Selective A₃ adenosine receptor antagonist radioligand for human and rodent species. ACS Med. Chem. Lett. 2022, in press, https://doi.org/10.1021/acsmedchemlett.1c00685.
(50) van Galen, P. J. M.; Nissen, P.; van Wijngaarden, I.; IJzerman, A. P.; Soudijn, W. ¹H-imidazo[4,5-c]quinolin-4-amines: novel non-xanthine adenosine antagonists. J. Med. Chem. 1991, 34, 1202-1206.
(51) Draper-Joyce, C. J., Bhola, R., Wang, J. et al. Positive allosteric mechanisms of adenosine A₁ receptor-mediated analgesia. Nature 597, 571-576 (2021). https://doi.org/10.1038/s41586-021-03897-2
(52) Jacobson, M. P.; Pincus, D. L.; Rapp, C. S.; Day, T. J. F.; Honig, B.; Shaw, D. E.; Friesner, R. A., “A Hierarchical Approach to All-Atom Protein Loop Prediction,” Proteins: Structure, Function and Bioinformatics, 2004, 55, 351-367.
(53) Jacobson, M. P.; Friesner, R.A.; Xiang, Z.; Honig, B., “On the Role of Crystal Packing Forces in Determining Protein Sidechain Conformations,” J. Mol. Biol., 2002, 320, 597-608.
(54) Schrödinger Release 2021-1: Schrödinger, LLC, New York, NY, 2021.
(55) Sastry, G. M.; Adzhigirey, M.; Day, T.; Annabhimoju, R.; Sherman, W., “Protein and ligand preparation: Parameters, protocols, and influence on virtual screening enrichments,” J. Comput. Aid. Mol. Des., 2013, 27 (3), 221-234.
(56) Lu, C.; Wu, C.; Ghoreishi, D.; Chen, W.; Wang, L.; Damm, W.; Ross, G.; Dahlgren, M.; Russell, E.; Von Bargen, C.; Abel, R.; Friesner, R.; Harder, E.; OPLS4: Improving Force Field Accuracy on Challenging Regimes of Chemical Space, Journal of Chemical Theory and Computation 2021 17 (7), 4291-4300, DOI: 10.1021/acs.jctc.1c00302.
(57) Halgren, T., “Identifying and Characterizing Binding Sites and Assessing Druggability,” J. Chem. Inf. Model., 2009, 49, 377-389.
(58) Halgren, T., “New Method for Fast and Accurate Binding-site Identification and Analysis,” Chem. Biol. Drug Des., 2007, 69, 146-148.
(59) Farid, R.; Day, T.; Friesner, R. A.; Pearlstein, R. A., “New insights about HERG blockade obtained from protein modeling, potential energy mapping, and docking studies,” Bioorg. & Med. Chem., 2006, 14, 3160-3173.
(60) Sherman, W.; Day, T.; Jacobson, M. P.; Friesner, R. A.; Farid, R., “Novel Procedure for Modeling Ligand/Receptor Induced Fit Effects,” J. Med. Chem., 2006, 49, 534-553.
(61) Sherman, W.; Beard, H. S.; Farid, R., “Use of an Induced Fit Receptor Structure in Virtual Screening,” Chemical Biology & Drug Design, 2006, 67, 83-84.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I), a pharmaceutically acceptable salt thereof, or a stereoisomer thereof, 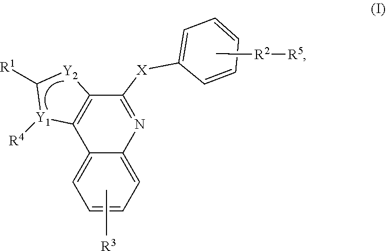
wherein
R¹ is selected from the group consisting of alkyl, cycloalkyl, bicycloalkyl, tricycloalkyl, alkenyl, cycloalkenyl, bicycloalkenyl, tricycloalkenyl, cycloalkoxyalkyl, hydroxycycloalkyl, and cycloalkylcarbonyl, each of which is optionally deuterated, or optionally halogenated; and
R² is absent, halo, dihalo, trihalo, SF₅, or a substituent selected from the following groups, each of which is optionally deuterated: alkylenyl, alkylaminocarbonyl, alkenyl, alkylamino carbonyl alkenyl, hetero bicyclylamino alkyl, carbonyl alkenyl, alkynyl, alkylaminoalkyl alkynyl, heterocyclylaminoalkyl alkynyl, heterocyclyloxy alkynyl, alkylaminocarbonylalkenyl, alkylamino iminoalkenyl, halo thiophenyl alkenyl, oxadizolylalkenyl, imidazolylaryl alkynyl, trialkoxyarylalkenyl, dioxyalrylalkenyl, monoalkoxyarylalkenyl, trifluoromethoxyarylalkenyl, trialkyl tin, and combinations thereof, wherein more than one R² group can be present on the phenyl ring, and wherein each of the R² group, other than halo, dihalo, trihalo, and SF₅, is optionally deuterated and/or halogenated;
R³ is hydrogen, deuterium, and/or halogen;
R⁴ is hydrogen or alkyl;
R⁵ is absent, halo, or a substituent selected from the group consisting of alkoxycarbonyl, which is optionally deuterated, amino, alkylamino, dialkylamino, trialkylamino, tetraalkylammonio, Boc-amino, guanidino, heterocyclylamino, hetero bicyclylamino, alkylamino imino, SF₅, halo thiophenyl, oxadizolyl, imidazolylaryl, trialkoxyaryl, dioxyaryl, monoalkoxyaryl, trifluoromethoxyaryl, trialkyl tin, and combinations thereof, each of which is optionally be substituted with a reporter group, linked through an amine, amide, triazole, sulfonamide, urea, or thiourea group,
X is O, S, or NR, wherein R is H or alkyl; and
Y₁ and Y₂ are independently N or NH, O, or S;
wherein the alkyl group is C₁-C₁₂ linear or branched alkyl, the cycloalkyl group is C₃-C₁₀ cycloalkyl group, the cyclic group of the bi- or tri-cycloalkyl is 3-10 membered, the alkenyl group is C₂-C₁₂ linear or branched alkenyl group, the cycloalkenyl group is 3-10 membered cyclic alkenyl group, the bicycloalkenyl and the tricycloalkenyl groups have 4 to 10 membered cycloalkenyl rings, the cycloalkoxyalkyl group is a C₃-C₁₀ cycloalkoxy C₁-C₁₂ linear or branched cycloalkyl group, the hydroxycycloalkyl group includes a hydroxyl group on a C₃-C₁₀ cycloalkyl group, and the cycloalkylcarbonyl group includes a C₃-C₁₀ cycloalkyl group.

2. The compound of claim 1, a pharmaceutically acceptable salt thereof, or a stereoisomer thereof, wherein X is NH, R² is 3,4-dichloro or 4-iodo, R³ is H, Y₁ and Y₂ are N, R⁴ is H, and R¹ is selected from the group consisting of

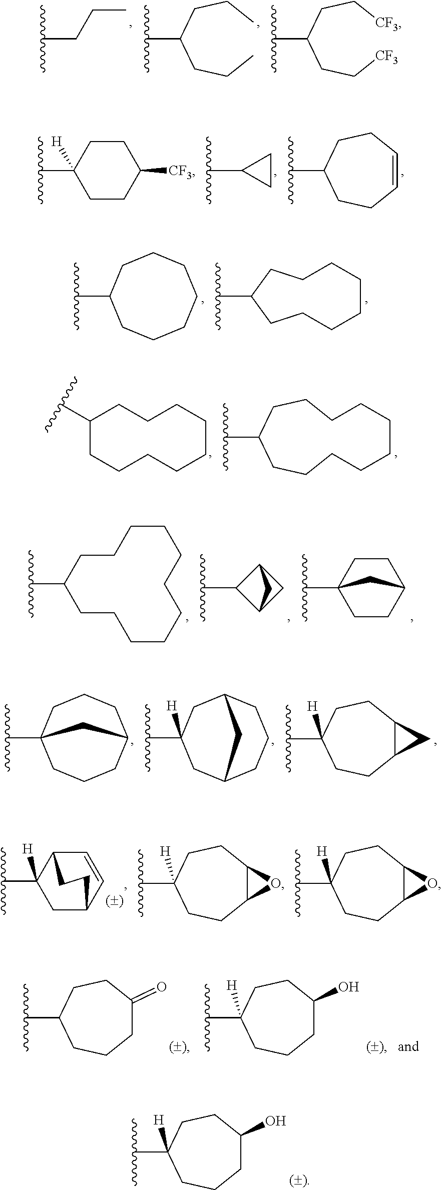

3. The compound of claim 1, a pharmaceutically acceptable salt thereof, or a stereoisomer thereof, wherein X is NH, R² is 4-iodo, 4-bromo, 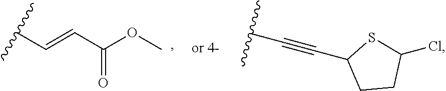
R¹ is cyclohexyl or 4-hept-4-yl, R³ is H, Y₁ and Y₂ are N, and R⁴ is H.

4. The compound of claim 1, a pharmaceutically acceptable salt thereof, or a stereoisomer thereof, wherein the compound is one of the following:

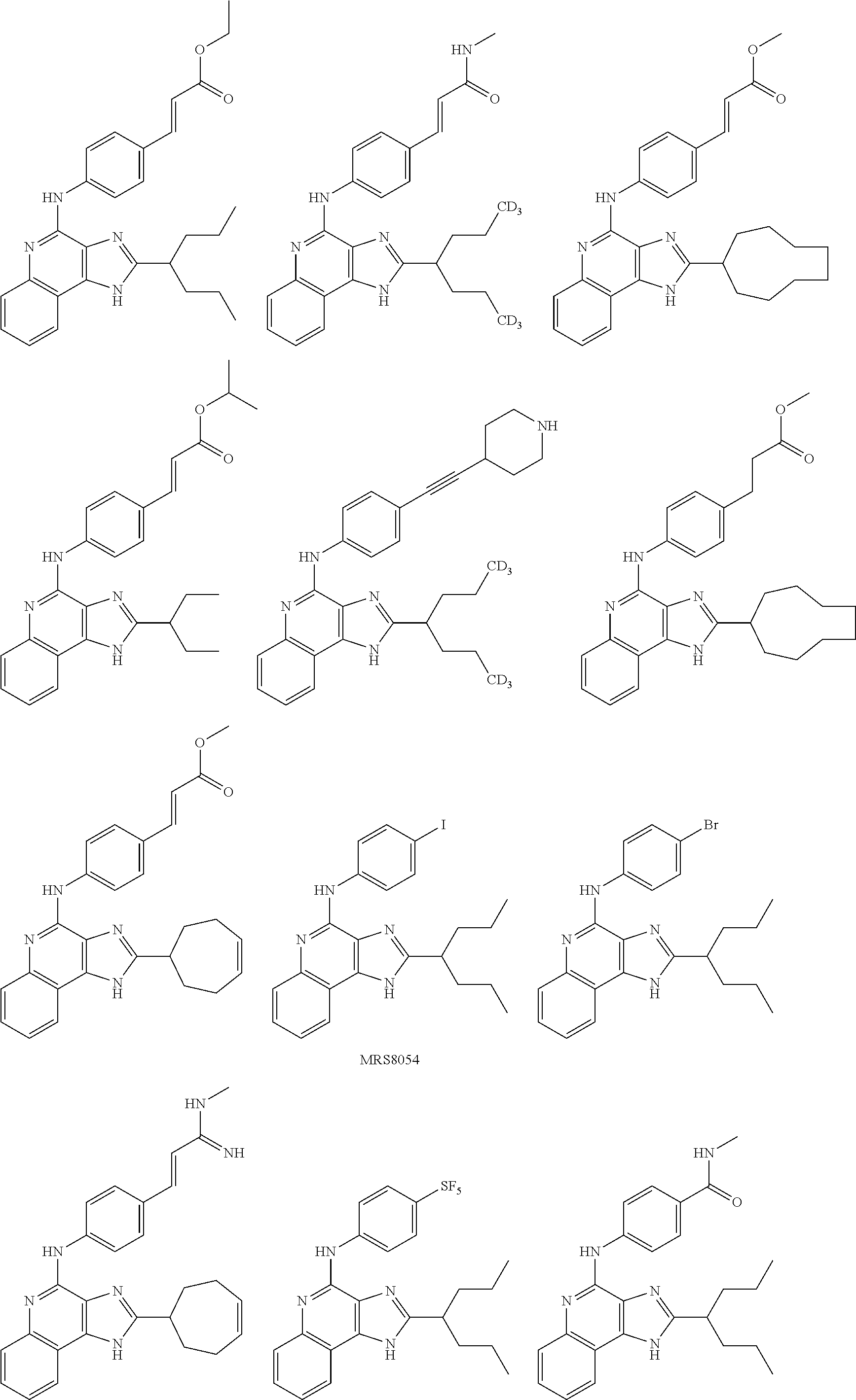

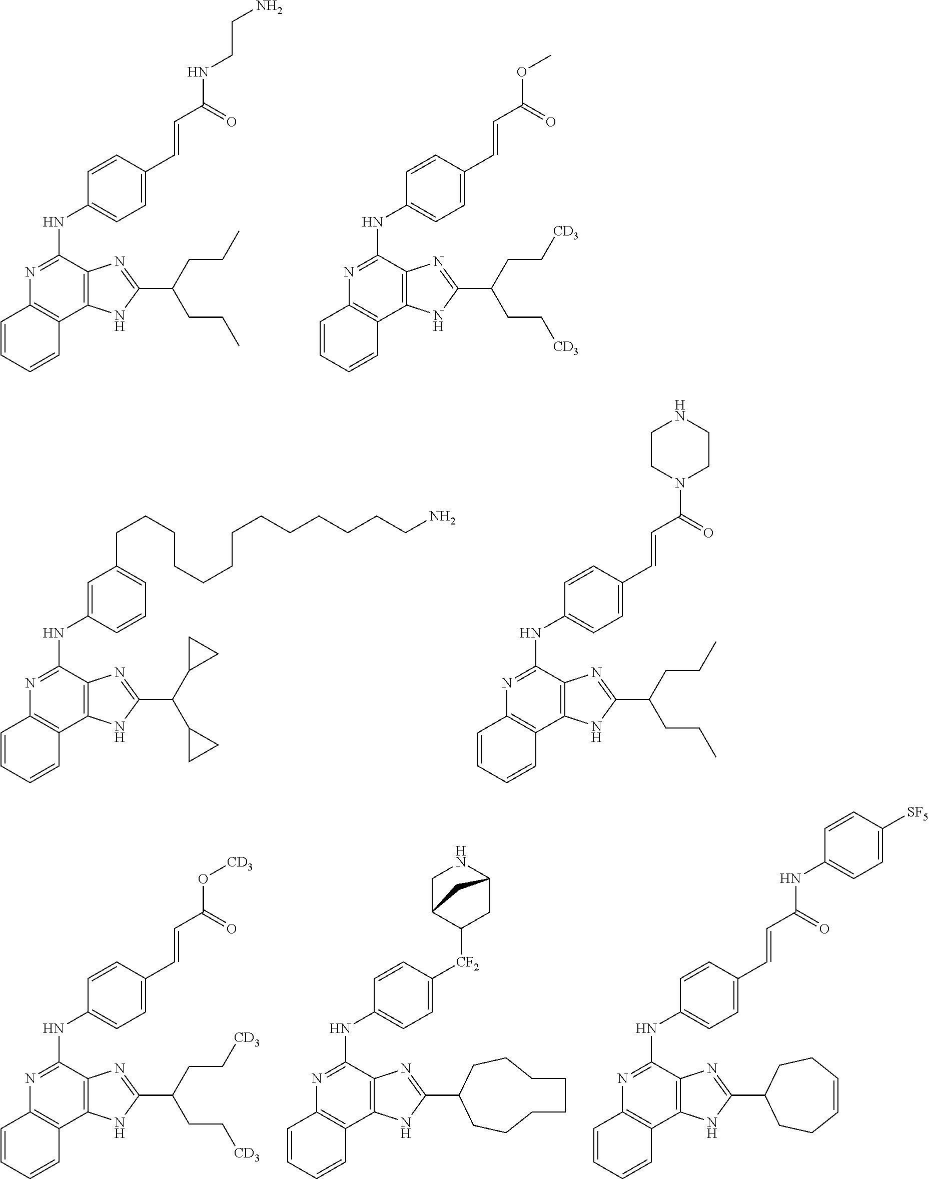

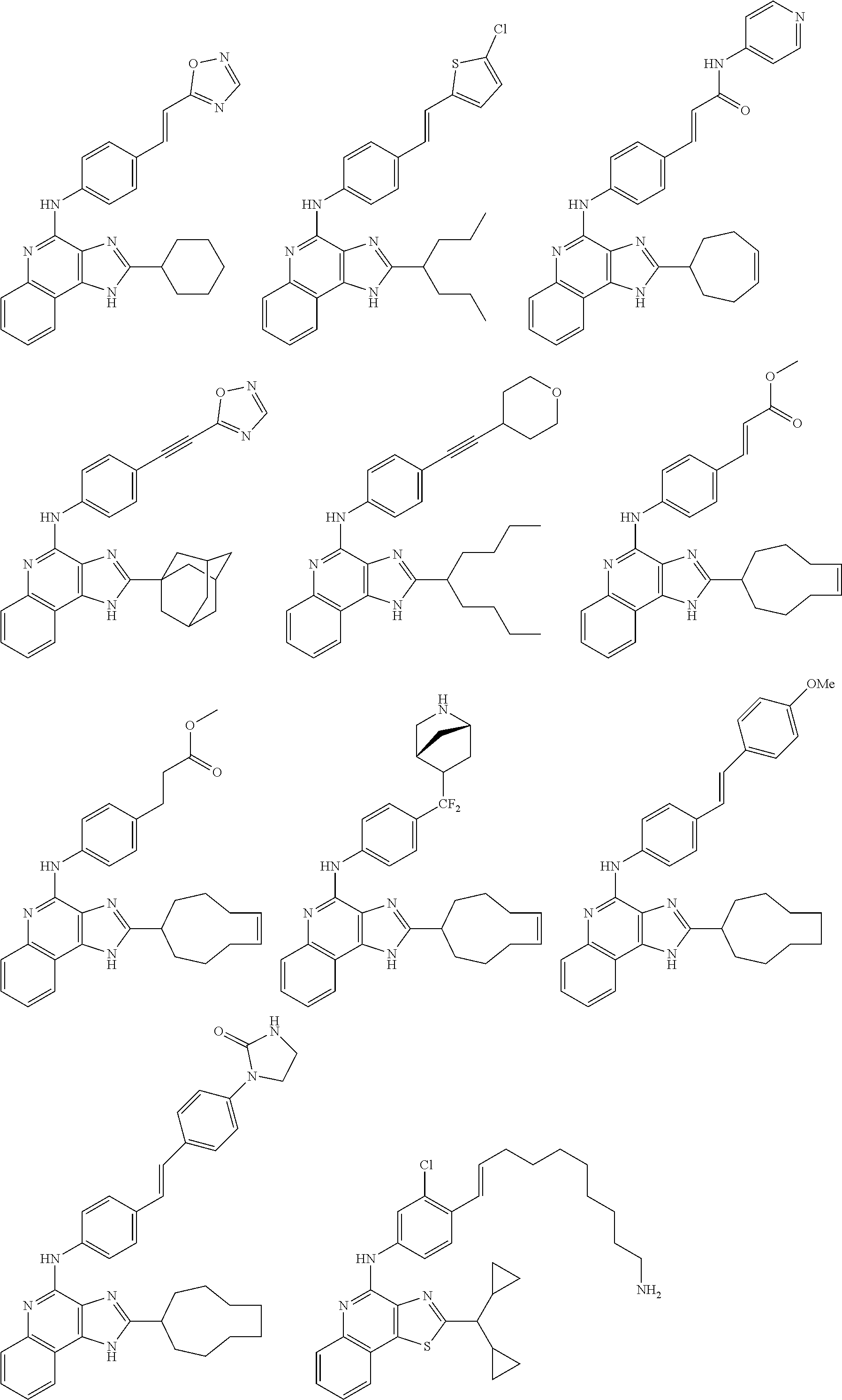

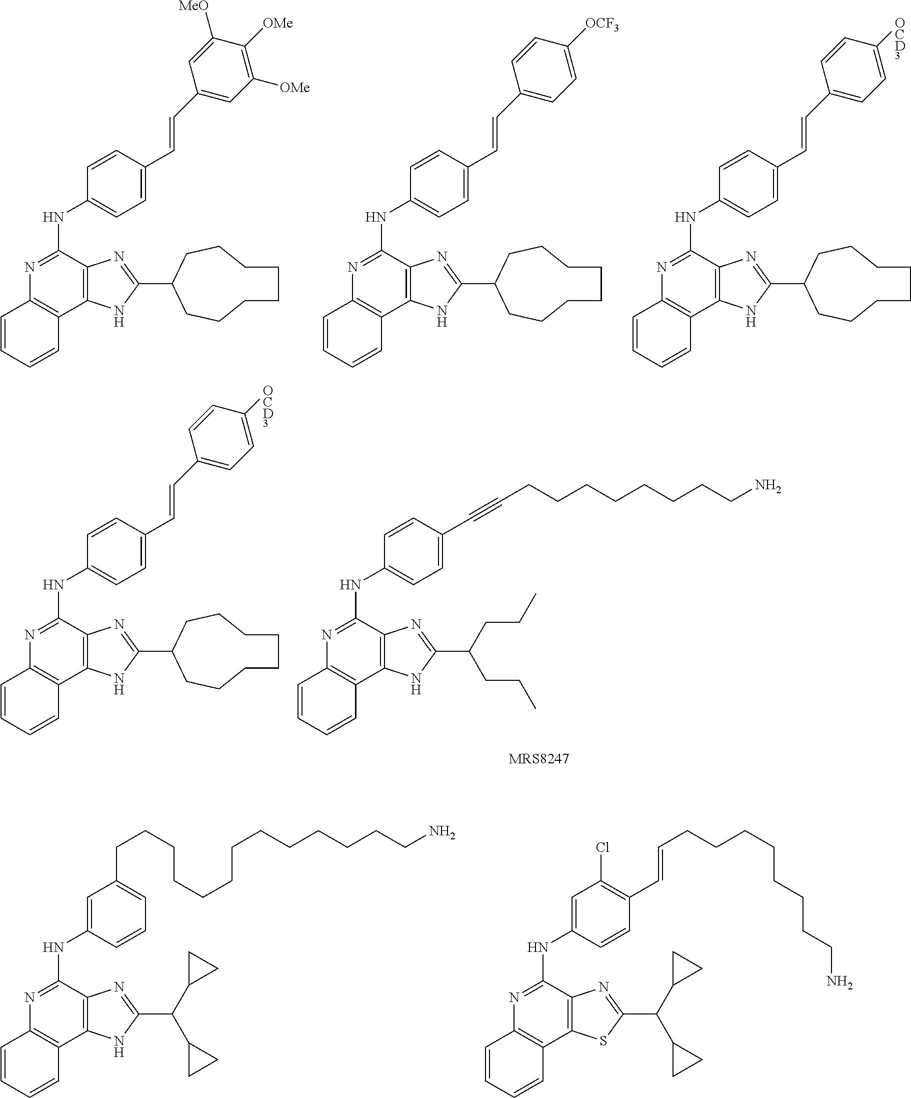

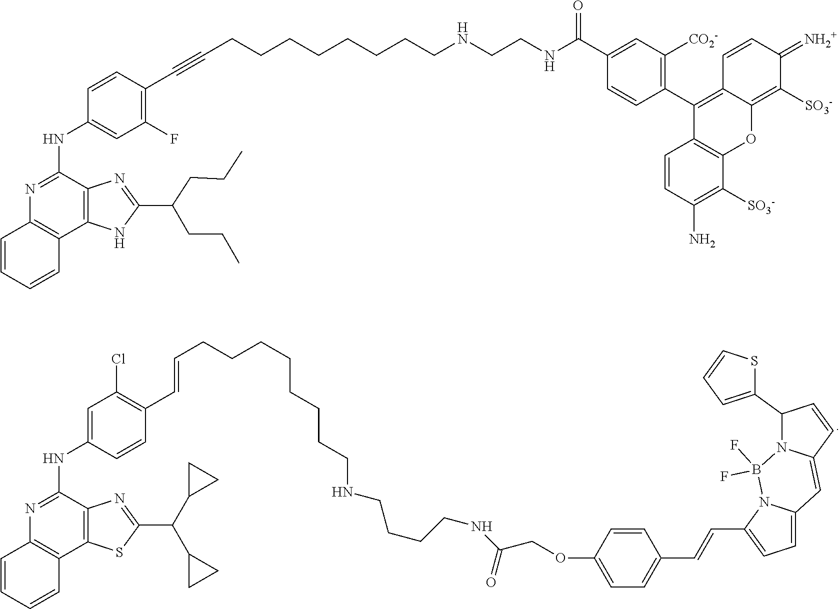

5. The compound of claim 1, a pharmaceutically acceptable salt thereof, or a stereoisomer thereof, wherein the reporter group is a biotin moiety or fluorescent dye.

6. The compound of claim 1, a pharmaceutically acceptable salt thereof, or a stereoisomer thereof, wherein X is NH, Y₁ and Y₂ are N or NH—, R² is aminoalkylamino carbonyl alkenyl, wherein the aminoalkyl moiety is —(CH₂)₂—NH₂ or (CH₂)₈—NH₂ or wherein R² is alkylamino carbonyl alkenyl, wherein the alkyl moiety is —(CH₂)₅—CH₃, and R³ and R⁴ are H.

7. The compound of claim 1, a pharmaceutically acceptable salt thereof, or a stereoisomer thereof, wherein X is NH, Y₁ and Y₂ are N or NH, and R² is aminoalkyl alkynyl or alkylaminoalkyl alkynyl, which could be attached at o, m, or p-position of the phenyl ring, and R³ and R⁴ are H.

8. The compound of claim 7, a pharmaceutically acceptable salt thereof, or a stereoisomer thereof, wherein R² is p-(CH₂)₈—NH₂, p-(CH₂)₆—NH₂, p-(CH₂)₄—NH₂, p-(CH₂)₈—NHCH₃, or m-(CH₂)₈—NH₂.

9. The compound of claim 1, a pharmaceutically acceptable salt thereof, or a stereoisomer thereof, wherein X is NH, Y₁ and Y₂ are N or NH, and R² aminoalkyl alkenyl or aminoalkyl, and R³ and R⁴ are H.

10. The compound of claim 9, a pharmaceutically acceptable salt thereof, or a stereoisomer thereof, wherein R² is —CH═CH—(CH₂)₈—NH₂ or —(CH₂)₁₀—NH₂.

11. The compound of claim 1, a pharmaceutically acceptable salt thereof, or a stereoisomer thereof, wherein X is NH, Y₁ and Y₂ are N or NH, R¹ is alkyl or cycloalkyl, R² mono or dihalo, and R³ and R⁴ are H.

12. The compound of claim 11, a pharmaceutically acceptable salt thereof, or a stereoisomer thereof, wherein R¹ is cyclohexyl, heptan-4-yl, nonan-5-yl, or di(cyclopropylmethyl)CH—, and R² is 3,4-dichloro or 4-iodo, and R⁵ is absent.

13. The compound of claim 1, a pharmaceutically acceptable salt thereof, or a stereoisomer thereof, wherein X is O, S, or NH, Y₁ and Y₂ are N or NH, R¹ is alkyl or cycloalkyl, R² mono or dihalo, R³ is H, and R⁴ is H, alkyl, arylalkyl, or hydroxyalkyl, and R⁵ is absent.

14. The compound of claim 13, a pharmaceutically acceptable salt thereof, or a stereoisomer thereof, wherein R¹ is heptan-4-yl, cyclohexyl, nonan-5-yl, or di(cyclopropylmethyl)CH—, and R² is H, R³ is H, and R⁴ is methyl, ethanyl-1-ol, benzyl, 3,4-dichloro, 4-iodo, or 3-chloro-4-iodo.

15. The compound of claim 1, a pharmaceutically acceptable salt thereof, or a stereoisomer thereof, wherein X is NH, Y₁ is N, Y₂ is S or O, R¹ is cyclohexyl, R² is 3,4-dichloro, R⁵ is absent, and R³ and R⁴ are H.

16. The compound of claim 1, a pharmaceutically acceptable salt thereof, or a stereoisomer thereof, wherein X is O, S, or NR, wherein R is methyl, Y₁ and Y₂ are N or NH, R¹ is cyclohexyl, R² is 3,4-dichloro, R⁵ is absent, and R³ and R⁴ are H.

17. The compound of claim 1, a pharmaceutically acceptable salt thereof, or a stereoisomer thereof, wherein the compound is

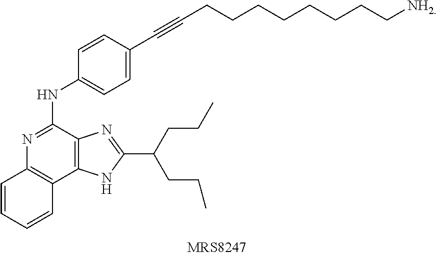 [SMILES: CCCC(CCC)c1nc2c(Nc3ccc(C#CCCCCCCCCN)cc3)nc3ccccc3c2[nH]1]

18. A pharmaceutical composition comprising a compound, salt, or stereoisomer of claim 1 and a pharmaceutically acceptable carrier.

19. The pharmaceutical composition of claim 18, wherein the pharmaceutically acceptable carrier is one that forms an emulsion, hydrogel, liposome, liquid nanoparticle, and/or micelle in combination with the compound, a pharmaceutically acceptable salt thereof, or a stereoisomer thereof.

20. A method of treating a subject for a condition which responds to modulation of A₃ adenosine receptor (A₃AR), the method comprising administering an effective amount of a compound, salt, or stereoisomer of claim 1.

21. The method of claim 20, wherein the subject has a condition selected from the group consisting of chronic neuropathic pain, heart disease, suppressed immunity, disease of the liver, psoriasis, and cancer.

22. (canceled)

23. A method of preparing a compound of claim 1, the method comprising:
(i) nitrating a dihydroxy compound of the formula 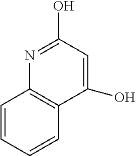 [SMILES: Oc1cc(O)c2ccccc2n1]
by reaction with concentrated nitric acid to obtain a nitrated dihydroxy compound of the formula 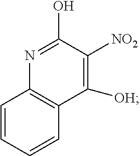 [SMILES: O=[N+]([O-])c1c(O)nc2ccccc2c1O]
(ii) converting the hydroxy groups of the compound from step (i) to chloro groups by reaction with a phosphorous oxychloride at an elevated temperature to obtain a compound of the formula: 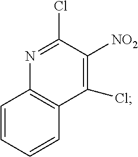 [SMILES: O=[N+]([O-])c1c(Cl)nc2ccccc2c1Cl]
(iii) reacting the compound from (ii) with a composition comprising aqueous ammonia to obtain the monochloro compound of the formula: 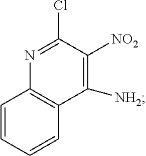 [SMILES: Nc1c([N+](=O)[O-])c(Cl)nc2ccccc12]
(iv) reducing the nitro group of the compound obtained in (iii) to obtain a diamine of the formula: 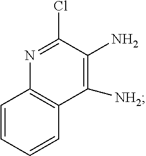 [SMILES: Nc1c(Cl)nc2ccccc2c1N]
(v) reacting the compound from (iv) with R¹—COOH to obtain a compound of the formula: 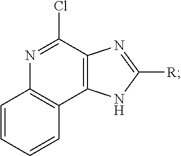
(vi) reacting the compound from (v) with an amine to obtain a compound of formula: [chemical structure]
